(19) United States
(12) Patent Application Publication
(10) Pub. No.: US 2020/0255877 A1
(43) Pub. Date: Aug. 13, 2020

(54) MICROBIAL PRODUCTION OF NICOTINAMIDE RIBOSIDE

(71) Applicant: DSM IP ASSETS B.V., Heerlen (NL)

(72) Inventors: Adam LAWRENCE, Kaiseraugst (CH); Celine VIAROUGE, Kaiseraugst (CH)

(21) Appl. No.: 16/614,563

(22) PCT Filed: May 17, 2018
(86) PCT No.: PCT/EP2018/062979

§ 371 Date: Nov. 18, 2019

(51) Int. Cl. C12P 19/30; C12N 1/14

(52) U.S. Cl. CPC C12P 19/30; C12N 2510/02; C12N 1/14

(57) ABSTRACT

The present invention is directed to microbial production of nicotinamide riboside and/or nicotinamide mononucleotide using a genetically modified fungus.

### CROSS-REFERENCE TO RELATED APPLICATIONS

This application is the U.S. national phase of International Application No. PCT/EP2018/062979 filed 17 May 2018 which designated the U.S. and claims the benefit of US Provisional Application No. 62/508,284 filed 18 May 2017, and to US Provisional Application No. 62/525,137 filed 26 Jun. 2017, the entire contents of each of which are hereby incorporated by reference.

### FIELD

The present invention is directed to microbial production of nicotinamide riboside and/or nicotinamide mononucleotide using a genetically modified fungus.

### BACKGROUND AND SUMMARY

Nicotinamide riboside (NR) is a pyridine-nucleoside form of vitamin B3 that functions as a precursor to nicotinamide adenine dinucleotide (NAD+). It is believed that high dose nicotinic acid can help to elevate high-density lipoprotein cholesterol, lowers low-density lipoprotein cholesterol and lower free fatty acids, although its mechanism has not been completely understood. NR has been synthesized chemically in the past. The biological pathways leading to the synthesis of NR are known but producing NR at an industrial level remains a challenge.

The chemical synthesis of nicotinamide mononucleotide (NMN) is similarly challenging, and current methods require expensive reagents and enzymatic catalysis. Like NR, the biological pathways are known, but have not been able to be engineered for industrial production.

Thus, it is desirable to identify new methods for producing NR and/or NMN more efficiently, in particular for biotechnological production of NR and/or NMN.

Surprisingly, the inventors have now found a novel method for significantly increasing the production rate of nicotinamide ribose and/or nicotinamide mononucleotide and created expression vectors and host cells useful in such methods.

In particular, the present invention is directed to a genetically modified fungal strain capable of converting nicotinic acid mononucleotide (NaMN) to nicotinamide mononucleotide (NMN), used for production of NR and/or NMN, wherein said strain comprising nicotinic acid mononucleotide amidating protein (NadE*) activity.

### BRIEF DESCRIPTION OF THE DRAWINGS

FIG. 1 represents the biochemical pathway for synthesizing quinolinate from aspartate and dihydroxyacetone phosphate in the presence of NadA and NadB enzymes;

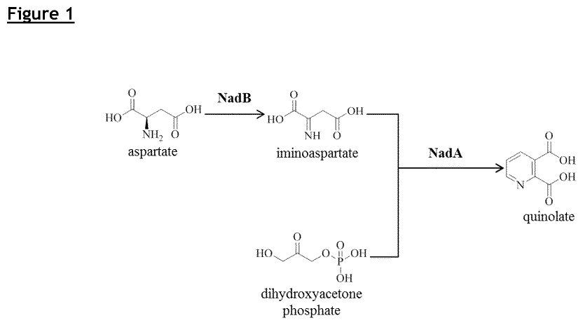

FIG. 2 represents the biochemical pathways and enzymes for synthesizing nicotinamide adenine dinucleotide;

FIG. 3 represents the biochemical pathways useful for the production of nicotinamide riboside from NAD+ or intermediates of NAD+ biosynthesis; and

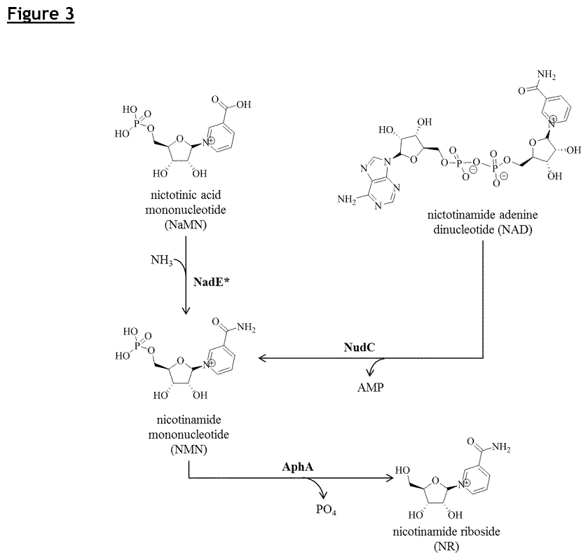

FIG. 4 represents the biochemical pathways with undesirable activities for nicotinamide riboside production.

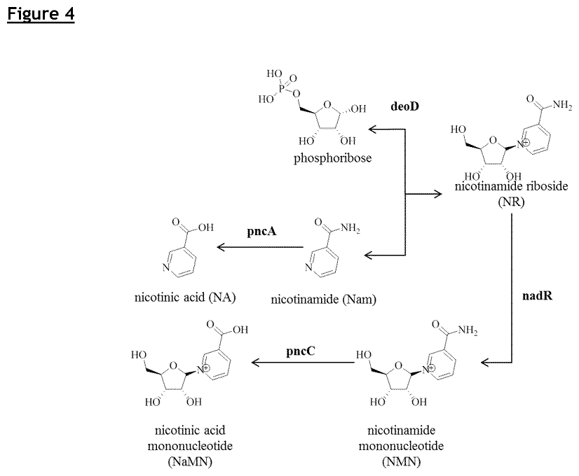

### DETAILED DESCRIPTION

The genetically modified fungal strain according to the present invention may further comprise one or more additional modifications including one or more modification(s) being selected from the group consisting of:

(a) introduction/expressing of a gene encoding a polypeptide having L-aspartate oxidase activity, in particular wherein the gene is of bacterial origin;

(b) introduction/expressing of a gene encoding a polypeptide having quinolinate synthase activity, in particular wherein the gene is of bacterial origin;

(c) reducing nicotinamide riboside transporter protein activity;

(d) reducing nicotinic acid mononucleotide adenyltransferase activity;

(e) reducing nicotinamide riboside kinase activity;

(f) reducing purine nucleoside phosphorylase activity; and

(g) modifying the activity of nicotinamide mononucleotide hydrolase activity.

Thus, a genetically modified fungal strain according to the present invention comprises a polynucleotide sequence encoding a polypeptide having NadE* activity, said polynucleotide being in particular of bacterial origin. Thus, the fungal strain is modified via introduction of a heterologous gene, in particular a bacterial origin, wherein the heterologous gene encodes a polypeptide having NadE* activity and wherein the production of NR and/or NMN in such fungal strain is increased compared to the production of said compounds using the respective wild-type fungal strain.

The gene encoding polypeptide having NadE* activity which is used for the purpose of the present invention might be originated from any bacterial source, including but not limited to a microorganism selected from the group consisting of Francisella, Dichelobacter, Mannheimia, and Actinobacillus, such as e.g. Francisella tularensis, Francisella sp. FSC1006, Francisella guangzhouensis, Francisella sp. TX077308, Francisella philomiragia, Francisella noatunensis, Francisella persica, Francisella cf. novicida 3523, Francisella tularensis, Dichelobacter nodosus, Mannheimia succinoproducens, or Actinobacillus succinogenes, in particular selected from the group consisting of F. tularensis, F. sp. FSC1006, F. guangzhouensis, F. sp. TX077308, F. philomiragia subsp. philomiragia ATCC 25017, F. philomiragia strain 0#319-036 [FSC 153], F. noatunensis supbsp. orientalis str. Toba 04, F. philomiragia strain GA01-2794, F. persica ATCC VR-331, F. cf. novicida 3523, F. tularensis subsp. novicida D9876, F. tularensis subsp. novicida F6168, F. tularensis subsp. tularensis strain NIH B-38, F. tularensis subsp. holarctica F92, Dichelobacter nodosus VCS1703A, Mannheimia succinoproducens MBEL55E, and Actinobacillus succinogenes.

Preferably, the polypeptide having NadE* activity comprises an amino acid sequence with at least 50%, 60%, 70%, 80%, 90%, 95%, 97%, 99% or 100% identity to an amino acid sequence selected from a sequence according to SEQ ID NO: 1, 3, 4, 5, 6, 7, 8, 9, 10, 11, 12, 13, 14, 15, 16, 17 or 18, wherein said polypeptide has a nicotinic acid amidating activity for converting nicotinic acid mononucleotide to nicotinamide mononucleotide. Said polypeptide might be encoded by a polynucleotide including a sequence according to SEQ ID NO: 2, 25, 26, 24, 22, 27, 23, 20, 19, 21, 37, 34, 35, 33, 31, 36, 32, 29, 28, 30, 38, 39.

In particular, a polypeptide having NadE* activity is selected from one of the following sequences according to Table 1.

TABLE 1
list of NadE* amino acids sequences

| SEQ ID No. | origin | Accession no. |
|---|---|---|
| 1 | Francisella tularensis FtNadE* | YP_170217 |
| 3 | Francisella sp. FSC1006 | WP_040008427.1 |
| 4 | Francisella guangzhouensis 08HL01032 | WP_039124332.1 |
| 5 | Francisella sp. TX077308 | WP_013922810.1 |
| 6 | Francisella philomiragia subsp. philomiragia | WP_004287429.1 |
| | ATCC 25017 | |
| 7 | Francisella philomiragia strain O#319-036 | WP_042517896.1 |
| | [FSC 153] | |
| 8 | Francisella noatunensis supbsp. orientalis | WP_014714556.1 |
| | str. Toba 04 | |
| 9 | Francisella philomiragia strain GA01-2794 | WP_044526539.1 |
| 10 | Francisella persica ATCC VR-331 | WP_064461307.1 |
| 11 | Francisella cf. novicida 3523 | WP_014548640.1 |
| 12 | Francisella tularensis subsp. novicida D9876 | WP_003037081.1 |
| 13 | Francisella tularensis subsp. novicida F6168 | WP_003034444.1 |
| 14 | Francisella tularensis subsp. tularensis strain | WP_003025712.1 |
| | NIH B-38 | |
| 15 | Francisella tularensis subsp. holarctica F92 | WP_010032811.1 |
| 16 | Dichelobacter nodosus VCS1703A | WP_011927945.1 |
| 17 | Mannheimia succinoproducens MBEL55E | WP_011201048.1 |
| 18 | Actinobacillus succinogenes | WP_012072393.1 |

According to an embodiment of the present invention, such modified strain as described herein which is able to convert NaMN to NMN, is used in a process for producing NR. In particular, a process according to the present invention is comprising culturing said strain under conditions effective to produce NR and recovering NR from the medium.

Thus, the present invention is related to a process for production of NR, comprising:

(a) culturing a genetically modified fungal strain capable of the conversion of NaMN to NMN as described herein under conditions effective to produce NR,

(b) recovering NR from the medium,

wherein the fungal strain is encoding a heterologous polypeptide having NadE* activity.

In one embodiment, the genetically modified fungal strain as described herein furthermore comprises a heterologous gene encoding a protein having L-aspartate oxidase activity, preferably wherein the gene is originated from bacteria, including a gene comprising a nucleic acid sequence encoding a polypeptide according to SEQ ID NO: 41 or 42.

In another embodiment, the genetically modified fungal strain as described herein furthermore comprises a heterologous gene encoding a protein having quinolinate synthase activity, preferably wherein the gene is originated from bacteria, including a gene comprising a nucleic acid sequence encoding a polypeptide according to SEQ ID NO: 77, 78, or 79. This modification might be combined with further modification, e.g. the introduction/expression of a heterologous gene encoding a protein having L-aspartate oxidase activity as described herein.

In another embodiment, the genetically modified fungal strain as described herein furthermore comprises a reduction in nicotinamide riboside transporter protein activity, such as e.g. mutation in the endogenous gene encoding nicotinamide riboside transporter protein activity, including a gene comprising a nucleic acid sequence encoding a polypeptide according to SEQ ID NO: 62, 63 or 64. This modification might be combined with further modification, e.g. the introduction/expression of a heterologous gene encoding a protein having L-aspartate oxidase activity and/or introduction/expression of a heterologous gene encoding a protein having quinolinate synthase activity as described herein.

In a further embodiment, the genetically modified fungal strain as described herein furthermore comprises a reduction in nicotinic acid mononucleotide adenyltransferase activity, such as e.g. mutation in the endogenous gene encoding nicotinic acid mononucleotide adenyltransferase activity, including a gene comprising a nucleic acid sequence encoding a polypeptide according to SEQ ID NO: 51, 52, 53, 54, 55, 56 or 57. This modification might be combined with further modification, e.g. the introduction/expression of a heterologous gene encoding a protein having L-aspartate oxidase activity and/or introduction/expression of a heterologous gene encoding a protein having quinolinate synthase activity and/or reduction of endogenous nicotinamide riboside transporter protein activity as described herein.

In a further embodiment, the genetically modified fungal strain as described herein furthermore comprises a reduction in nicotinamide ribose kinase activity, such as e.g. mutation in the endogenous gene encoding nicotinamide ribose kinase activity, including a gene comprising a nucleic acid sequence encoding a polypeptide according to SEQ ID NOs: 68, 69 or 70. This modification might be combined with further modification, e.g. the introduction/expression of a heterologous gene encoding a protein having L-aspartate oxidase activity and/or introduction/expression of a heterologous gene encoding a protein having quinolinate synthase activity and/or reduction of endogenous nicotinamide riboside transporter protein activity and/or reduction of endogenous nicotinic acid mononucleotide adenyltransferase activity as described herein.

In a further embodiment, the genetically modified fungal strain as described herein furthermore comprises a reduction in purine nucleoside phosphorylase activity, such as e.g. mutation in the endogenous gene encoding purine nucleoside phosphorylase activity, including a gene comprising a nucleic acid sequence encoding a polypeptide according to SEQ ID NO: 71 or 72. This modification might be combined with further modification, e.g. the introduction/expression of a heterologous gene encoding a protein having L-aspartate oxidase activity and/or introduction/expression of a heterologous gene encoding a protein having quinolinate synthase activity and/or reduction of endogenous nicotinamide riboside transporter protein activity and/or reduction of endogenous nicotinic acid mononucleotide adenyltransferase activity and/or reduction of endogenous in nicotinamide riboside kinase activity as described herein.

Depending on the use of the fungal strain according to the present invention, the strain might carry a further modification in nicotinamide mononucleotide hydrolase activity.

In one embodiment, the genetically modified fungal strain as described herein furthermore comprises a modification in nicotinamide mononucleotide hydrolase activity, such as e.g. modification in the endogenous gene encoding nicotinamide mononucleotide hydrolase activity, including a gene comprising a nucleic acid sequence encoding a polypeptide according to SEQ ID NO: 43, 44, 45, 46, 47, 48, 49, 50 or it is originated from bacteria, preferably a gene comprising a nucleic acid sequence encoding a polypeptide according to SEQ ID NO: 73, 74, or 75. This modification might be combined with further modification, e.g. the introduction/expression of a heterologous gene encoding a protein having L-aspartate oxidase activity and/or introduction/expression of a heterologous gene encoding a protein having quinolinate synthase activity and/or reduction of endogenous nicotinamide riboside transporter protein activity and/or reduction of endogenous nicotinic acid mononucleotide adenyltransferase activity and/or reduction of endogenous in nicotinamide riboside kinase activity and or reduction of endogenous purine nucleoside phosphorylase activity as described herein. Such strain is in particular useful for production of NMN.

Thus in yet another embodiment, the genetically modified fungal strain as described herein furthermore comprises modified nicotinamide mononucleotide hydrolase activity, such as e.g. introduction/expression of a heterologous gene encoding nicotinamide mononucleotide hydrolase activity, preferably originated from bacteria, including a gene comprising a nucleic acid sequence encoding a polypeptide according to SEQ ID NO: 43, 44, 45, 46, 47, 48, 49, 50 or is originated from bacteria, preferably a gene comprising a nucleic acid sequence encoding a polypeptide according to SEQ ID NO: 73, 74, or 75, or wherein the activity of the endogenous gene/polypeptide is increased compared to the respective wild-type strain. This modification might be combined with further modification, e.g. the introduction/expression of a heterologous gene encoding a protein having L-aspartate oxidase activity and/or introduction/expression of a heterologous gene encoding a protein having quinolinate synthase activity and/or reduction of endogenous nicotinamide riboside transporter protein activity and/or reduction of endogenous nicotinic acid mononucleotide adenyltransferase activity and/or reduction of endogenous in nicotinamide riboside kinase activity and or reduction of endogenous purine nucleoside phosphorylase activity as described herein. Such strain is in particular useful for production of NMN.

According to the present invention, the introduction of a gene encoding a polypeptide having NadE* activity, said polynucleotide being in particular of bacterial origin, results in increased production of NR and/or NMN using said fungal strain. Preferably, the polypeptide having NadE* activity is chosen from the ones listed in Table 1.

In a further aspect, the present invention is directed to a genetically modified fungal strain capable of converting nicotinic acid mononucleotide (NaMN) to nicotinamide mononucleotide (NMN), said strain comprising a polypeptide having NadE* activity, said polypeptide having at least 50%, 60%, 70%, 80%, 90%, 95%, 97%, 99% or 100% identity to an amino acid sequence selected from a sequence according to any one of SEQ ID NO: 1, 3, 4, 5, 6, 7, 8, 9, 10, 11, 12, 13, 14, 15, 16, 17 or 18 and comprising a tyrosine at position 27 and/or a glutamine at position 133, and/or a arginine at position 236 in SEQ ID NO: 1, based on the ClustalW method of alignment when compared to SEQ ID NOS: 1 and 3 to 18 using the default parameters of GAP PENALTY=10, GAP LENGTH PENALTY=0.1, and Gonnet 250 series of protein weight matrix. The result of the sequence alignment is shown in FIG. 5.

A suitable fungal strain to be genetically modified according to the present invention can be any fugus, such as including but not limited to Saccharomyces, Yarrowia, Aspergillus, Pichia, Kluyveromyces, Pichia, Ashbya, in particular Saccharomyces cerevisiae, Yarrowia lipolytica, Aspergillus niger, Pichia pastoris, Kluyveromyces lactic, Ashbya gossypii, preferably Saccharomyces or Yarrowia.

In one particular embodiment, the genetically modified fungal strain is S. cerevisiae expressing a polypeptide having NadE* activity, such as e.g. a polypeptide according to any one of SEQ ID NO: 1, 3, 4, 5, 6, 7, 8, 9, 10, 11, 12, 13, 14, 15, 16, 17 or 18, in particular encoded by the FtNadE* or gene or its functional homologs resulting in production of excess NMN. Excess NMN can be exported and converted to NR by native NMN nucleosidase.

As used herein, scientific and technical terms used herein will have the meanings that are commonly understood by one of ordinary skill in the art.

The term NadE* or “polypeptide having NadE* activity” is used interchangeably herein and indicates an enzyme capable of catalyzing the conversion of NaMN to NMN.

The term “quinolinate synthase” indicates an enzyme capable of converting iminosuccinic acid and dihydroxyacetone phosphate to quinolinate and phosphate. The quinolinate synthase used in this invention can be from various organisms, such as E. coli, B. subtilis, C. glutamicum, etc. Examples of quinolinate synthase proteins include polypeptides having amino acid sequence according to SEQ ID NO:76, 77, or 78. Genes encoding the quinolinate synthesis activity are provided under, for example, accession nos. ACX40525 (E. coli), NP_390663 (B. subtilis), and CAF19774 (C. glutamicum). The quinolinate synthase as defined includes functional variants of the above mentioned quinolinate synthases.

The term “L-aspartate oxidase” indicates an enzyme capable of converting aspartic acid to iminosuccinic acid in an FAD dependent reaction. The L-aspartate oxidase used in this invention can be from various organisms, such as E. coli, B. subtilis, C. glutamicum, etc. Examples of nucleoside hydrolase proteins include polypeptides having amino acid sequence SEQ ID NO: 41 or 42. Genes encoding the L-aspartate oxidase activity are provided under, for example, accession nos. ACX38768 (E. coli) and NP_390665 (B. subtilis). The L-aspartate oxidase as defined includes functional variants of the above-mentioned L-aspartate oxidases.

The term “nicotinamide riboside transporter protein” indicates an enzyme capable of catalyzing the transport of nicotinamide riboside for importing nicotinamide riboside from the periplasm to the cytoplasm. The enzyme in S. cerevisiae is known as NRT1. The nicotinamide riboside transporter protein described in this invention is a native polypeptide of the host organism such as S. cerevisiae, A. niger, Y. lipolytica, etc. Examples of nicotinamide riboside transporter proteins include polypeptides having amino acid sequences SEQ ID NO: 62, 63 or 64. Genes encoding the NR transport activity are provided under, for example, accession nos. CAG67923 (A. baylyi), NP_599316 (C. glutamicum), NP_415272 (E. coli), and WP_003227216.1 (B. subtilis).

The term “nicotinamide mononucleotide phosphorylase” indicates an enzyme capable of catalyzing the phosphorolysis of nicotinamide mononucleotide to nicotinamide riboside. The enzyme in S. cerevisiae is known as PNP1. The nicotinamide mononucleotide phosphorylase protein described in this invention can also be a native polypeptide of the host organism such as S. cerevisiae, A. niger, Y. lipolytica, etc. Examples of nucleoside phosphorylase proteins include polypeptides having amino acid sequence SEQ ID NOs: 71, 72.

The term “nucleoside hydrolase” or “nicotinamide mononucleotide nucleosidase” indicates an enzyme capable of catalyzing the conversion of nicotinamide mononucleotide to nicotinamide riboside. The nucleoside hydrolase used in this invention can be from various organisms, such as E. coli, B. subtilis, C. glutamicum etc. The nucleoside hydrolase protein described in this invention can also be a native polypeptide of the host organism such as S. cerevisiae, A. niger, Y. lipolytica, etc. Examples of nucleoside hydrolase proteins include polypeptides having amino acid sequence SEQ ID NOs: 43, 44, 45, 46, 47, 48, 49, 50, 73, 74 or 75. Genes encoding the nucleoside phosphorylase activity are provided under, for example, accession nos. NP_415013 (E. coli), NP_388665 (B. subtilis), and CAF18899 (C. glutamicum).

The term “nicotinic acid mononucleotide adenyltransferase” indicates an enzyme capable of catalyzing the conversion of nicotinic acid mononucleotide to nicotinic acid adenine dinucleotide. The enzymes in S. cerevisiae is known as NMA1 and NMA2. The nicotinic acid mononucleotide adenyltransferase protein described in this invention is a native polypeptide of the host organism such as S. cerevisiae, A. niger, Y. lipolytica, etc. Examples of nicotinic acid mononucleotide adenyltransferase proteins include polypeptides having amino acid sequences SEQ ID NOs: 51, 52, 53, 54, 55, 56 or 57.

The relatedness between two amino acid sequences or between two nucleotide sequences is described by the parameter “sequence identity”. For purposes of the present disclosure, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

The term “nucleic acid construct” means a nucleic acid molecule, either single or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure.

The term “control sequences” means all components necessary for the expression of a polynucleotide encoding a polypeptide of the present disclosure. Each control sequence may be native or foreign to the polynucleotide encoding the polypeptide or native or foreign to each other. Such control sequences include, but are not limited to, a leader, polyadenylation sequence, peptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “operably linked” means a configuration in which a control sequence is placed at an appropriate position relative to the coding sequence of a polynucleotide such that the control sequence directs the expression of the coding sequence.

The term “expression” includes any step involved in the production of the polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to additional nucleotides that provide for its expression.

The term “host cell” means any fungal cell type that is susceptible to transformation, transfection, transduction, and the like with a nucleic acid construct or expression vector comprising a polynucleotide encoding any one of the polypeptide sequences of the present disclosure. The term “host cell” encompasses any progeny of a parent cell that is not identical to the parent cell due to mutations that occur during replication.

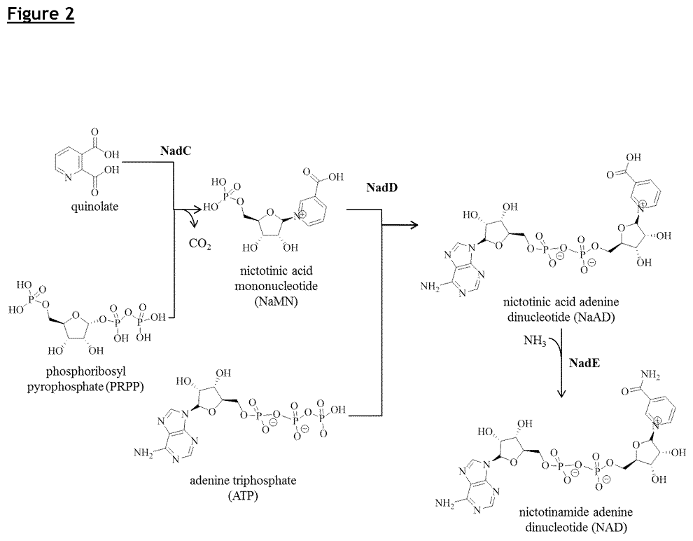

The nadE gene product from E. coli, B. subtilis, and most characterized bacterial species, as well as all characterized eukaryotic species, utilizes nicotinic acid adenine dinucleotide as substrate for an amidation reaction to produce NAD+. By this native pathway, nicotinamide riboside (NR) is obtained by breakdown of nicotinamide adenine dinucleotide (NAD+), as in previously described work (U.S. Pat. No. 8,114,626 B2) or according to FIG. 2.

The host fungal cell may be genetically modified by any manner known to be suitable for this purpose by the person skilled in the art. This includes the introduction of the genes of interest, such as the gene encoding the nicotinic acid amidating protein NadE*, into a plasmid or cosmid or other expression vector which are capable of reproducing within the host cell. Alternatively, the plasmid or cosmid DNA or part of the plasmid or cosmid DNA or a linear DNA sequence may integrate into the host genome, for example by homologous recombination or random integration. To carry out genetic modification, DNA can be introduced or transformed into cells by natural uptake or by well-known processes such as electroporation. Genetic modification can involve expression of a gene under control of an introduced promoter. The introduced DNA may encode a protein which could act as an enzyme or could regulate the expression of further genes.

Genetic modification of a microorganism can be accomplished using classical strain development and/or molecular genetic techniques. Such techniques known in the art and are generally disclosed for microorganisms, for example, in Sambrook et al., 1989, Molecular Cloning: A Laboratory Manual, Cold Spring Harbor Labs Press. The reference Sambrook et al., ibid., is incorporated by reference herein in its entirety.

Suitable vectors for construction of such an expression vector are well known in the art and may be arranged to comprise the polynucleotide operably linked to one or more expression control sequences, so as to be useful to express the required enzymes in a host cell, for example a fungal cell as described above. For example, promoters including, but not limited to, T7 promoter, pLac promoter, nudC promoter, ushA promoter, pVeg promoter can be used in conjunction with endogenous genes and/or heterologous genes for modification of expression patterns of the targeted gene. Similarly, exemplary terminator sequences include, but are not limited to, the use of XPR1, XPR2, CPC1 terminator sequences.

As used herein, the term “specific activity” or “activity” with regards to polypeptides as described means its catalytic activity, i.e. its ability to catalyze formation of a product from a given substrate. The specific activity defines the amount of substrate consumed and/or product produced in a given time period and per defined amount of protein at a defined temperature. As used herein “reduction of activity” means to reduce the total quantity of an enzyme in a cell or to reduce the specific activity of said enzyme in order to effect a reduction in units of activity per unit biomass. As used herein “increased activity” means to increase the total quantity of an enzyme in a cell or to increase the specific activity of said enzyme in order to effect an increase in units of activity per unit biomass. A reduction of activity includes a total blocking or knocking out of the gene and/or polypeptide to activity which are reduced by at least 50, 25, 10% compared to a wild-type strain, wherein said gene/polypeptide is not modified.

As used herein, the production rate of NR and/or NMN is increased if the genetically modified fungal strain is capable of producing at least more than 20, 30, 50, 70, 80, 100% and more compared to a non-modified or wild-type fungal strain.

As used herein, “functional variant” means that the variant sequence has similar or identical functional enzyme activity characteristics to the enzyme having the native amino acid sequence specified herein. For example, a functional variant of SEQ ID NOs: 1 and 3 to 18 has similar or identical nicotinic acid amidating protein FtNadE* activity characteristics as SEQ ID NOs:1 and 3 to 18, respectively. An example may be that the rate of conversion by a functional variant of SEQ ID NOs: 1 and 3 to 18, of nicotinic acid mononucleotide to nicotinamide mononucleotide, may be the same or similar, although said functional variant may also provide other benefits. For example, at least about 80%, 90%, 95%, 96%, 97%, 98%, 99% or 100% the rate will be achieved when using the enzyme that is a functional variant of SEQ ID NOs: 1 and 3 to 18, respectively.

A functional variant or fragment of any of the above amino acid sequences, therefore, is any amino acid sequence which remains within the same enzyme category (i.e., has the same EC number). Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type. By “conservative substitution” is meant the substitution of an amino acid by another amino acid of the same class, in which the classes are defined as follows:

Class Amino Acid Examples:

Nonpolar: A, V, L, I, P, M, F, W

Uncharged polar: G, S, T, C,Y, N, Q

Acidic: D, E

Basic: K, R, H.

Nicotinamide ribose compounds produced according to the present disclosure can be utilized in any of a variety of applications, for example, exploiting their biological or therapeutic properties (e.g., controlling low-density lipoprotein cholesterol, increasing high-density lipoprotein cholesterol, etc.). For example, according to the present disclosure, nicotinamide ribose may be used in pharmaceuticals, foodstuffs, and dietary supplements, etc.

The nicotinamide riboside produced by the method disclosed in this invention could have therapeutic value in improving plasma lipid profiles, preventing stroke, providing neuroprotection with chemotherapy treatment, treating fungal infections, preventing or reducing neurodegeneration, or in prolonging health and well-being. Thus, the present invention is further directed to the nicotinamide riboside compounds obtained from the genetically modified fungal cell described above, for treating a disease or condition associated with the nicotinamide riboside kinase pathway of NAD+ biosynthesis by administering an effective amount of a nicotinamide riboside composition. Diseases or conditions which typically have altered levels of NAD+ or NAD+ precursors or could benefit from increased NAD+biosynthesis by treatment with nicotinamide riboside include, but are not limited to, lipid disorders (e.g., dyslipidemia, hypercholesterolaemia or hyperlipidemia), stroke, neurodegenerative diseases (e.g., Alzheimer's, Parkinsons and Multiple Sclerosis), neurotoxicity as observed with chemotherapies, Candida glabrata infection, and the general health declines associated with aging. Such diseases and conditions can be prevented or treated by diet supplementation or providing a therapeutic treatment regime with a nicotinamide riboside composition.

It will be appreciated that, the nicotinamide riboside compounds isolated from the genetically modified fungal strains of this invention can be reformulated into a final product. In some other embodiments of the disclosure, nicotinamide riboside compounds produced by manipulated host cells as described herein are incorporated into a final product (e.g., food or feed supplement, pharmaceutical, etc.) in the context of the host cell. For example, host cells may be lyophilized, freeze dried, frozen or otherwise inactivated, and then whole cells may be incorporated into or used as the final product. The host cell may also be processed prior to incorporation in the product to increase bioavailability (e.g., via lysis).

In some embodiments of the disclosure, the produced nicotinamide riboside compounds are incorporated into a component of food or feed (e.g., a food supplement). Types of food products into which nicotinamide riboside compounds can be incorporated according to the present disclosure are not particularly limited, and include beverages such as milk, water, soft drinks, energy drinks, teas, and juices; confections such as jellies and biscuits; fat-containing foods and beverages such as dairy products; processed food products such as rice, bread, breakfast cereals, or the like. In some embodiments, the produced nicotinamide riboside compound is incorporated into a dietary supplement, such as, for example, a multivitamin.

The following examples are intended to illustrate the invention without limiting its scope in any way.

### EXAMPLES

All basic molecular biology and DNA manipulation procedures described herein are generally performed according to Sambrook et al., Ausubel et al. or Barth. (J. Sambrook, E.F. Fritsch, T. Maniatis (eds). 1989. Molecular Cloning: A Laboratory Manual. Cold Spring Harbor Laboratory Press: New York; F. M. Ausubel, R. Brent, R. E. Kingston, D. D. Moore, J. G. Seidman, J. A. Smith, K. Struhl (eds.). 1998. Current Protocols in Molecular Biology. Wiley: New York); and G. Barth (ed.). 2013. Yarrowia lipolytica: Biotechnological Applications. Springer Science Et Business Media, Berlin.

### Example 1

### Identification of Sequences Coding for NaMN Amidating Activity (NadE*)

Sorci and co-workers identified the enzyme FtNadE* encoded by the genome of Francisella tularensis (SEQ ID NO:1) and demonstrated its ability to function both in vivo and in vitro as a nicotinamide mononucleotide (NaMN) amidating enzyme (Sorci L. e., 2009). In addition, they proposed that three amino acid residues were responsible for the enzyme's substrate preference for NaMN over NaAD: Y27; Q133; and R236. In order to identify additional sequences encoding this function, 50 unique nucleotide sequences derived from a BLAST search of the NCBI nr/nt database on 14 Sep 2016 using default parameters for tBlastn with the amino acid sequence for FtNadE (SEQ ID NO:2) were translated and aligned using the Geneious alignment algorithm (Biomatters, LLLC.). 16 of these sequences had a conserved tyrosine, glutamine and arginine which aligned with Y27, Q133 and R236, respectively (i.e. contained a “Y-Q-R motif”) and were predicted to encode NaMN amidating enzymes (SEQ ID NOs: 3 to 18 and).

### Example 2

### Genetic Constructs for Expression of NaMN Amidating Activity (NadE*) in E. coli

10 sequences encoding predicted nadE* open reading frames (SEQ ID NOs:2 and 19 to 27) were selected based on maximizing phylogenetic distance among the set of 16 predicted nadE* genes and were codon optimized for expression in E. coli using the Geneious codon optimization algorithm with the E. coli K-12 codon usage table and threshold to be rare set at 0.4. The optimized sequences (SEQ ID NOs 28 to 37) were synthesized de novo by GenScript, Inc., and cloned into XhoI/NdeI digested pET24a(+) (Novagen, Inc.), also by GenScript, yielding the plasmids in Table 2. Plasmids were transformed into BL21(DE3), allowing for IPTG induction of the nadE* genes in order to induce NR synthesis and yielding the strains ME407, ME644, ME645, ME646, ME647, ME648, ME649, ME650, ME651, ME652 (Table 3).

TABLE 2
Plasmids used in this study

| plasmid | Description |
|---|---|
| pET24Ft | SEQ ID No. 37 (FtNadE*) cloned in pET24a(+) |
| pET24Dn | SEQ ID No. 29 (DnNadE*) cloned in pET24a(+) |
| pET24As | SEQ ID No. 30 (AsNadE*) cloned in pET24a(+) |
| pET24Fph | SEQ ID No. 31 (FphNadE*) cloned in pET24a(+) |
| pET24Fn | SEQ ID No. 32 (FnNadE*) cloned in pET24a(+) |
| pET24FspT | SEQ ID No. 33 (FspTNadE*) cloned in pET24a(+) |
| pET24FspF | SEQ ID No. 34 (FspFNadE*) cloned in pET24a(+) |
| pET24Fg | SEQ ID No. 35 (FgNadE*) cloned in pET24a(+) |
| pET24Fpe | SEQ ID No. 36 (FpeNadE*) cloned in pET24a(+) |
| pET24Mn | SEQ ID No. 28 (MnNadE*) cloned in pET24a(+) |
| pET24Ft-TGV | SEQ ID No. 42 (FtNadE*-TGV) cloned in pET24a(+) |
| pETMn-TGV | SEQ ID No. 43 (MnNadE*-TGV) cloned in pET24a(+) |
| pETFn-TGV | SEQ ID No. 44 (FnNadE*-TGV) cloned in pET24a(+) |
| pETFspT-TGV | SEQ ID No. 45 (FspTNadE*-TGV) cloned in pET24a(+) |
| pET-EcNadE | SEQ ID No. 46 (EcNadE) cloned in pET24a(+) |
| pBS-FnNadE | SEQ ID No. 38 cloned into pUC57 |
| pBS-FtNadE | SEQ ID No. 40 cloned into pUC57 |
| pBS-FspNadE | SEQ ID No. 41 cloned into pUC57 |
| pBS-MnNadE | SEQ ID No. 39 cloned into pUC57 |
| MB4124-FnNadE | SEQ ID No. 47 cloned into MB4124 |

TABLE 3
Strains used or described in this study

| Strain | Species | Genotype |
|---|---|---|
| BL21(DE3) | E. coli | fhuA2 [Ion] ompT gal (λ DE3) |
| | | [dcm] ΔhsdS λ DE3 = λ |
| | | sBamHIo ΔEcoRI-B int::(lacI::PlacUV5::T7 |
| | | gene1) I21 Δnin5 |
| ME407 | E. coli | BL21(DE3) pET24Ft |
| ME409 | E. coli | BL21(DE3) |
| ME644 | E. coli | BL21(DE3) pET24Dn |
| ME645 | E. coli | BL21(DE3) pET24As |
| ME646 | E. coli | BL21(DE3) pETFph |
| ME647 | E. coli | BL21(DE3) pET24 Fn |
| ME648 | E. coli | BL21(DE3) pET24FspT |
| ME649 | E. coli | BL21(DE3) pET24FspF |
| ME650 | E. coli | BL21(DE3) pET24Fg |
| ME651 | E. coli | BL21(DE3) pET24Fpe |
| ME652 | E. coli | BL21(DE3) pET24Mn |
| ME708 | E. coli | BL21(DE3) pETFn-rev |
| ME710 | E. coli | BL21(DE3) pETFspT-rev |
| ME712 | E. coli | BL21(DE3) pET24Ft-rev |
| ME714 | E. coli | BL21(DE3) pETMn-rev |
| ME683 | E. coli | BL21(DE3) pETEcNadE |
| BS168 | B. subtilis | Wildtype strain |
| BS6209 | B. subtilis | BS168 nadR::spec |
| ME479 | B. subtilis | BS168 deoD::tet |
| ME492 | B. subtilis | BS168 pupG::neo |
| ME496 | B. subtilis | BS168 nadR::spec deoD::tet |
| ME517 | B. subtilis | BS168 nadR::spec deoD::tetpupG::neo |
| ME795 | B. subtilis | ME517 amyE::cat pVeg-MsNadE* |
| ME805 | B. subtilis | ME517 amyE::cat pVeg-rbs4-FnNadE* |
| ME820 | B. subtilis | ME517 amyE::cat pVeg-FspNadE* |
| ME824 | B. subtilis | ME517 amyE::cat pVeg-rbs4-FtNadE* |
| ATCC13032 | C. glutamicum | Wildtype strain |
| ME763 | C. glutamicum | ATCC13032 MB4124-FnNadE |

### Example 3

### Characterization of E. coli Strains Expressing NadE* Enzymes

To test the effect of NadE* expression on NR production, E. coli strains were inoculated from single colonies into LB medium and grown overnight (2 mL, 37° C., 15 mL test tube, 250 rpm, 50 μg/mL kanamycin). Precultures (200 μL) were used to inoculate 2 mL M9nC medium with or without 25 μM IPTG and grown in 24 well deep well plates (Whatman Uniplate, 10 mL, round bottom) sealed with an AirPore tape sheet (Qiagen) for three days (Infors Multitron Shaker, 800 rpm, 80% humidity). Samples were analyzed by LC-MS as described herein. Without plasmid, NR production was below the limit of quantification in the presence and absence of induction with 25 μM IPTG. Strains harboring plasmids for expression of NadE* enzymes produced up to 2.7 mg/L NR upon induction (Table 4).

TABLE 4
Nicotinamide riboside concentrations (mg/L) in E. coli shake
plate cultures upon IPTG induction (average of 2 cultures)

| Strain | Enzyme | No IPTG | 25 μm IPTG |
|---|---|---|---|
| ME407 | FtNadE* | <LOQ | 0.11 |
| ME409 | None | <LOQ | <LOQ |
| ME644 | DnNadE* | <LOQ | 0.28 |
| ME645 | AsNadE* | 0.08 | 0.31 |
| ME646 | FphNadE* | 0.02 | <LOQ |
| ME647 | FnNadE* | <LOQ | 1.91 |
| ME648 | FspTNadE* | 0.03 | 1.29 |
| ME649 | FspFNadE* | <LOQ | 0.82 |
| ME650 | FgNadE* | <LOQ | 0.64 |
| ME651 | FpeNadE* | 0.14 | 1.09 |
| ME652 | MnNadE* | 0.1 | 2.73 |

### Example 4

### Increased NR Production in E. coli Requires a NadE* with Y27, Q133 and R236

To demonstrate the importance of the YQR motif for NR production, four of the E. coli optimized NadE* sequences were altered to remove the residues which aligned to the Francisella tularensis Y27, Q133, R236 residues and replaced with the amino acid resides coded for in the Bacillus anthracis NadE (T, G, Et V, respectively; SEQ ID NOs: 42 to 45). Site directed mutagenesis of the corresponding pET24a(+) plasmids was performed by GenScript, Inc, resulting in the plasmids in Table 2. Plasmids were transformed into BL21(DE3), allowing for IPTG induction of the nadE-TGV genes and yielding the strains, ME708, ME710, ME712, and ME714 (Table 3). These strains with a NadE-TGV failed to exhibit similar IPTG dependent increases in NR production to strains with NadE* (Table 5).

TABLE 5
Nicotinamide riboside concentrations (mg/L) in
E. coli shake plate cultures upon IPTG induction.

| Strain | Enzyme | No IPTG | 50 μm IPTG |
|---|---|---|---|
| BL21(DE3) | none | <LOQ | <LOQ |
| BL21(DE3) | none | <LOQ | <LOQ |
| ME683 | EcNadE | <LOQ | <LOQ |
| ME683 | EcNadE | <LOQ | 0.01 |
| ME647 | FnNadE* | 0.04 | 0.92 |
| ME647 | FnNadE* | 0.02 | 0.75 |
| ME708 | FnNadE-TGV | <LOQ | 0.05 |
| ME708 | FnNadE-TGV | <LOQ | 0.07 |
| ME648 | FspTNadE* | <LOQ | 0.09 |
| ME648 | FspTNadE* | <LOQ | 0.16 |
| ME710 | FspTNadE-TGV | 0.01 | 0.01 |
| ME710 | FspTNadE-TGV | 0.02 | 0.01 |
| ME407 | FtNadE* | <LOQ | 0.1 |
| ME407 | FtNadE* | 0.02 | 0.12 |
| ME714 | FtNadE-TGV | 0.01 | <LOQ |
| ME714 | FtNadE-TGV | <LOQ | <LOQ |
| ME652 | MsNadE* | 0.07 | 1.11 |
| ME652 | MsNadE* | <LOQ | 0.96 |
| ME712 | MsNadE-TGV | <LOQ | 0.1 |
| ME712 | MsNadE-TGV | <LOQ | <LOQ |

### Example 5

### Overexpression of E. coli NadE is Not Sufficient to Observe Increased NR Production

To demonstrate that high levels of NaAD amidating activity (NadE) are insufficient to produce increased NR accumulation, the wildtype nadE open reading (SEQ ID NO: 46) frame was amplified via PCR from the genome of BL21(DE3) with primers M011159 and M011160 (Table 6) that added XhoI/NdeI restriction sites at the start and stop codons respectively. The PCR fragment was ligated into similarly digested pET24a(+), yielding plasmid pET24b+nadEBL21. This plasmid was transformed into BL21(DE3), allowing for IPTG induction of nadE and yielding the strain ME683. When tested for NR production alongside strains expressing NadE* sequences, this strain with additional expression of the E. coli NadE failed to exhibit IPTG dependent increases in NR concentration. (Table 5).

TABLE 6
Primers used in strain construction.

| | SEQ ID | | |
|---|---|---|---|
| Primer | NO: | Name | Use |
| 10444 | 79 | pDG1662_Pveg-I_pdxP | Amplification of all 3′ |
| | | Copy extraction (rev) | flanks |
| 10447 | 80 | amyE::nadEstar 5′ | Amplification of all |
| | | (reversed) (fwd) | 5′flanks |
| 11222 | 81 | amyE-FnNadE 3′ for | |
| 11223 | 82 | amyE-FnNadE 3′ rev | |
| 11226 | 83 | amyE-FspNadE 3′ for | |
| 11227 | 84 | amyE-FspNadE 3′ rev | |
| 11230 | 85 | amyE-MsNadE 3′ for | |
| 11231 | 86 | amyE-MsNadE 3′ rev | Amplification of MsNadE |
| | | | gBlock |
| 11232 | 87 | PvegI MsNadE 5′ amyE | Amplification of MsNadE |
| | | for | gBlock |
| 11233 | 88 | PvegI MsNadE 5′ amyE | Amplification of MsNadE |
| | | rev | 5′ flank |
| 11234 | 89 | amyE-FtNadE 3′ for | |
| 11235 | 90 | amyE-FtNadE 3′ rev | |
| 11341 | 91 | rbs4 FnNadE rev | |
| 11342 | 92 | rbs4 FnNadE For | |
| 11351 | 93 | pVegI-FspNadE Rev | |
| 11352 | 94 | pVegI-FspNadE For | |
| 11353 | 95 | rbs4 FtNadE rev | |
| 11354 | 96 | rbs4 FtNadE For | |
| 11159 | 97 | XhoI-3′ NadE BL21 | Amplification of EcNadE |
| 11160 | 98 | NdeI 5′-NadE-BL21 | Amplification of EcNadE |

### Example 6

### Construction and Characterization of Genetic Constructs Expressing the nadE* Genes in Y. lipolytica

This example describes the construction and characterization of genetic constructs expressing nadE* genes in Y. lipolytica. DNA fragments encoding the a NadE* protein, e.g. FnNadE*, are obtained by de novo DNA synthesis. Codon usage is optimized for expression in Y. lipolytica and DNA synthesis is carried out by GenScript, Inc. The nadE* gene is expressed in Y. lipolytica under control of a constitutive promoter. For example, the open reading frame for FnNadE* preceded by the sequence (gctagc)CACAAAA(atg) is cloned into NheI/MluI-digested pMB6157, resulting in plasmid p6157-FnNadE. Transformation into a strain such as ATCC201249 allows for constitutive expression of the nadE* gene in order to trigger NR or NMN synthesis.

For construction and Characterization of Genetic Constructs Expressing the nadA and nadB in Y. lipolytica, plasmids encoding genes for the synthesis of quinolinic acid are constructed as follows. Open reading frames for E. coli nadA and nadB are codon optimized for expression in Yarrowia lipolytica. The optimized sequences, are synthesized by GenScript, and amplified by PCR using appropriate primers flanked by 60 bp of the desired promoter and terminator. DNA fragments encoding promoter sequences, terminator sequences, or Yarrowia lipolytica markers are amplified by PCR from existing constructs. The vector, consisting of the S. cerevisiae centromere-based URA3 plasmid YCp50 (Gene. 1987; 60(2-3):237-43.) with ENOp, xprT, and LEU2 sequences from Yarrowia replacing the tet gene using standard techniques, is prepared from E. coli. All fragments are purified by gel electrophoresis using a QiaQuick kit (Qiagen). S. cerevisiae strain 10556-23C (W303 background; G. R. Fink) is transformed (Nat Protoc. 2007; 2(1):31-4.) with 250 ng of each DNA fragment and selected for prototrophy on minimal glucose aspartate medium. Plasmids are rescued from prototrophic transformants (Nucleic Acids Research, Vol. 20, No. 14, p. 3790 (1992)) and used to transform E. coli DH5a to ampicillin resistance (100 mg/L) on LB agar plates. Plasmids are digested with Sfil and used to transform a wildtype Yarrowia strain such as ATCC201249 to leucine prototrophy on minimal glucose aspartate medium containing adenine (0.2 mM). Prototrophic isolates are obtained by restreaking colonies from minimal media transformation plates to minimal media plates.

Y. lipolytica strains engineered for the production of NR are inoculated in YPD medium and grown for 3 days at 30° C.

### Example 7

### Construction and Characterization of Genetic Constructs Expressing the nadE* Genes in S. cerevisiae

This example describes the construction and characterization of genetic constructs expressing NadE* genes in S. cerevisiae. DNA fragments encoding the NadE* protein are obtained by de novo DNA synthesis and codon usage is optimized for expression in S. cerevisiae. DNA synthesis is carried out by GenScript, Inc. The nadE* gene is expressed in S. cerevisiae under control of a constitutive or inducible promoter. In order to introduce constructs for expression of the nadE* gene in S. cerevisiae, the open reading frame, promoter sequence, terminator sequence and marker sequence are amplified with primers which introduce 50 bp flanking sequences allowing for homologous recombination of the expression construct in the desired genomic location. For example, FnNadE* is operably linked to the Tef1 promoter, Adh1 terminator and CRE recognition site flanked KanMX and introduced to the genome at the NRK1 locus. Transformation into a strain such as Cen.PK allows for constitutive expression of the Fn-nadE* gene in order to trigger NR or NMN synthesis.

### Example 8

### Deletion of Genes in Saccharomyces cerevisiae

The genes encoding Nrt1, Nrk1, Pnp1 and Urh1 are deleted to render the host strain more competent for NR production under the manner of Brenner, et al (U.S. Pat. No. 8,114,626). Genes encoding NaMN and NMN adenyltransferases (Nma1, Nma2, Pof1) and nicotinic acid phosphoribosyltransferase (Npt1) are deleted to reduce competitive flux. Genes encoding NMN nucleosidase (Sdt1, Isn1) are deleted to increase production of NMN. The DNA fragment encoding KanMX, flanked by recognition sites from the CRE recombinase is amplified from the plasmid template with primers introducing 60 bp sequence homologous to the region 5′ and 3′ of the open reading frame to be removed. Yeast strain Cen.PK is transformed and selected for G418 resistance by plating to YPD agar with 200 pg/mL G418. Individual knockouts are confirmed by colony PCR with primers internal to KanMX and more than 60 bp 5′ or 3′ of the deleted open reading frame. Expression of the CRE recombinase in the strain so obtained renders the strain G418 sensitive, allowing for the deletion of multiple gene targets in a single strain by an iterative process.

S. cerevisiae strains engineered for the production of NR and/or NMN are inoculated in YPD medium and grown for 3 days at 30° C.

### Example 9

### Deletion of Nicotinamide Riboside in Production Cultures

NR is analyzed by liquid chromatography/mass spectrometry (LCMS). After cultivation, 100 μL is diluted in 900 μL MS diluent (10% Water 10mM Ammonium Acetate pH9.0, 90% acetonitrile) in 96 well deep well plates. Plates are centrifuged (10 min, 3000 rpm) and supernatant is transferred to a new plate for characterization. Supernatant is injected in 5 μl portions onto a HILIC UPLC column (Waters BEH Amide, 2.1×75 mm P/N 1860005657). Compounds are eluted at a flow rate of 400 μl min⁻¹, after a 1-minute hold, using a linear gradient from 99.9% (10 mM ammonium acetate at pH 9.0 with 95% acetonitrile/5% Water) mobile phase D, to 70% (10 mM ammonium acetate pH 9.0 50/50 Acetonitrile/Water) mobile phase C, over 12 minutes, followed by a 1-minute hold in mobile phase C, and 5 minutes re-equilibration in mobile phase D (FIG. 7). Eluting compounds are detected with a triple quadropole mass spectrometer using positive electrospray ionization. The instrument is operated in MRM mode and NR is detected using the transition m/z 123>80. NR is quantified by comparison to standard (Chromadex) injected under the identical condition. NMN is quantified by comparison to standard (Sigma Aldrich) injected under the identical condition.

1. A genetically modified fungal strain capable of converting nicotinic acid mononucleotide (NaMN) to nicotinamide mononucleotide (NMN), wherein said strain comprising nicotinic acid mononucleotide amidating protein (NadE*) activity.

2. A genetically modified fungal strain according to claim 1 which is selected from Saccharomyces, Yarrowia, Aspergillus, Pichia, Kluyveromyces, Pichia, Ashbya, preferably selected from Saccharomyces cerevisiae, Yarrowia lipolytica, Aspergillus niger, Pichia pastoris, Kluyveromyces lactic, Ashbya gossypii.

3. A genetically modified fungal strain according to claim 1 expressing a heterologous polypeptide with nicotinic acid mononucleotide amidating protein (NadE*) activity, said polypeptide being selected from bacterial source, preferably from Francisella, Dichelobacter, Mannheimia, and Actinobacillus.

4. A genetically modified fungal strain according to claim 1, wherein the polypeptide having NadE* activity comprises an amino acid sequence with at least 50%, 60%, 70%, 80%, 90%, 95%, 97%, 99% or 100% identity to an amino acid sequence selected from a sequence according to SEQ ID NO: 1, 3, 4, 5, 6, 7, 8, 9, 10, 11, 12, 13, 14, 15, 16, 17 or 18.

5. A genetically modified fungal strain according to claim 1, further comprising one or more additional modifications including one or more modification(s) being selected from the group consisting of:
(a) introduction/expressing of a gene encoding a polypeptide having L-aspartate oxidase activity, in particular wherein the gene is of bacterial origin;
(b) introduction/expressing of a gene encoding a polypeptide having quinolinate synthase activity, in particular wherein the gene is of bacterial origin;
(c) reducing nicotinamide riboside transporter protein activity;
(d) reducing nicotinic acid mononucleotide adenyltransferase activity;
(e) reducing nicotinamide riboside kinase activity;
(f) reducing purine nucleoside phosphorylase activity; and
(g) modifying the activity of nicotinamide mononucleotide hydrolase activity.

6. A genetically modified fungal strain according to claim 5, wherein the gene encoding a polypeptide having quinolinate synthase activity is originated from bacteria, preferably a gene comprising a nucleic acid sequence encoding a polypeptide according to SEQ ID NO: 76, 77, or 78.

7. A genetically modified fungal strain according to claim 5, wherein the gene encoding a polypeptide having nicotinamide riboside transporter activity which is to be reduced is comprising a nucleic acid sequence encoding a polypeptide according to SEQ ID NO: 62, 63 or 64.

8. A genetically modified fungal strain according to claim 5, wherein the gene encoding a polypeptide having L-aspartate oxidase activity is originated from bacteria, preferably a gene comprising a nucleic acid sequence encoding a polypeptide according to SEQ ID NO: 41 or 42.

9. A genetically modified fungal strain according to claim 5, wherein the gene encoding a polypeptide having nicotinamide mononucleotide hydrolase activity which is to be modified is comprising a nucleic acid sequence encoding a polypeptide according to SEQ ID NOs: 43, 44, 45, 46, 47, 48, 49, 50 or is originated from bacteria, preferably a gene comprising a nucleic acid sequence encoding a polypeptide according to SEQ ID NO: 73, 74, or 75.

10. A genetically modified fungal strain according to claim 5, wherein the gene encoding a polypeptide having nicotinic acid mononucleotide adenyltransferase activity which is to be reduced is comprising a nucleic acid sequence encoding a polypeptide according to SEQ ID NO: 51, 52, 53, 54, 55, 56 or 57.

11. A genetically modified fungal strain according to claim 5, wherein the gene encoding a polypeptide having purine nucleoside phosphorylase activity which is to be reduced is comprising a nucleic acid sequence encoding a polypeptide according to SEQ ID NO: 71 or 72.

12. A genetically modified fungal strain according to claim 5, wherein the gene encoding a polypeptide having nicotinamide ribose kinase activity which is to be reduced is comprising a nucleic acid sequence encoding a polypeptide according to SEQ ID NO: 68, 69 or 70.

13. A process for production of NMN, comprising:
(a) culturing a genetically modified fungal strain according to claim 1 under conditions effective to produce NMN,
(b) recovering NMN from the medium,
wherein the fungal strain is encoding a heterologous polypeptide having NadE* activity.

14. A process for production of NR, comprising:
(a) culturing a genetically modified fungal strain according to claim 1 under conditions effective to produce NR,
(b) recovering NR from the medium,
wherein the fungal strain is encoding a heterologous polypeptide having NadE* activity.


SEQUENCE LISTING (98 sequences)

SEQ ID NO: 1 - protein, 249 aa, Francisella tularensis
  MISC_FEATURE (1)..(249): Francisella tularensis NadE amino acid sequence
MKIVKDFSPKEYSQKLVNWLSDSCMNYPAEGFVIGLSGGIDSAVAASLAVKTGLPTTALILPSDNNQHQDMQDALELIEMLNIEHYTISIQPAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 2 - DNA, 750 bp, Francisella tularensis
  misc_feature (1)..(750): Francisella tularensis NadE nucleotide sequence
atgaaaatagttaaagattttagtcctaaagaatattcacaaaagttagttaattggctaagtgatagttgtatgaattatcctgctgaaggatttgtgattggtcttagtggcggtatagattcagcagttgcggcttctttagctgtcaaaactggattaccaactacagctttaatactaccttcagataataatcaacaccaagatatgcaagatgctctagagcttattgaaatgcttaatattgaacattataccatttcgattcaaccagcttatgaggcttttcttgcttcaacgcaaagctttacaaatctacaaaacaatagacaacttgtgatcaagggaaatgctcaagcacgtttaaggatgatgtatttgtatgcctatgcgcaacaatataacagaatagttataggtactgataatgcttgtgagtggtatatgggatattttacaaaattcggtgatggggctgccgatatacttccactagttaatctcaaaaaatctcaagtttttgaattaggcaaatacctagatgtccctaaaaacatacttgataaagctccatctgcaggactatggcaaggacaaactgatgaggatgaaatgggtgtaacttatcaagaaattgatgatttcttagatggtaaacaagtttcagcaaaagctctagaaagaataaatttctggcataatcgtagtcaccataagagaaaattagctttaactcctaatttctag

SEQ ID NO: 3 - protein, 249 aa, Francisella  sp.
  MISC_FEATURE (1)..(249): CP009574.1_translation Francisella sp. FSC1006, NadE
MSVVKNFKPNEYANKITEWLKDSCLNYPAEGFVVGISGGIDSAVAVSLAVNTGLPVTGLIMPSKNNDDKDTLDAIELAKKLNIEYHLIPIQPVYETFLDSAEDIKNSANDRQHVIKGNAQARFRMIYLYAYAQQNNRMVIGTDNACEWYMGYFTKFGDGAADILPLIKLKKSQVFELGSYLNVPNNILTKAPSAGLWLGQTDEAEMGVSYQEIDDFLDGKHVSDYALNQIKFWHNRSHHKRIMAKAPDF

SEQ ID NO: 4 - protein, 249 aa, Francisella guangzhouensis
  misc_feature (1)..(249): CP010427.1_translation Francisella guangzhouensis strain 08HL01032, NadE
MNVVKNFTPEKYSEKLIQWLTNSCIKYPAEGFVIGVSGGIDSAVCASLLSKTDLPTTAFILPSKNNSDQDMIDALELINKLNIPYHIIPIQPVYESFLKSTQLFTNPQNDRQNVIKGNAQARFRMMYLYAYAQQNNRIVVGTDNACEWYMGYFTKFGDGAADILPLINLKKSQVFELGKYLDVPRNILTKAPSAGLWQGQTDEGEMGVTYQEIDNFLDGKEVSPATFEKISYWHNRSHHKRKMALTPDF

SEQ ID NO: 5 - protein, 249 aa, Francisella sp.
  misc_feature (1)..(249): CP002872.1_translation Francisella sp. TX077308, NadE
MKIVKNFIVEQYSNNLIKWLKENCIKYPAEGFVIGISGGIDSAVAASLAVKTGLPTTALILPSKNNQDQDMRDGIELIENLNIEYHTVSIQPAYDTFIESTFNFTNSQNDRQHVIKGNAQARLRMMYLYAYAQQNNRIVIGTDNACEWYMGYFTKFGDGAADILPLINLKKSQVFELGKYLKVPKNIIQKDPSAGLWQGQTDEDEMGVTYKEIDDFLDGKEVSEKALERISFWHNRSHHKRSMAFTPNF

SEQ ID NO: 6 - protein, 249 aa, Francisella philomiragia
  misc_feature (1)..(249): CP000937.1_translation Francisella philomiragia subsp. philomiragia ATCC 25017, NadE
MKIIKNFIAEEYSKKLIEWLKKICINYPAEGFVIGISGGIDSAVAASLAVKTGLPTTALILPSKNNQDQDMKDGLELIKNLDIEHHIVPIQPAYDTFIESTLNFTNSQNDRQHVIKGNAQARLRMMYLYAYAQQNNRIVIGTDNACEWYMGYFTKFGDGAADILPLVNLKKSQVFEMGKYLKVPQNIIDKAPSAGLWQGQTDEDEMGVTYQEIDNFLDGKEVSAKALERINFWHNRSHHKRSMAFTPNF

SEQ ID NO: 7 - protein, 249 aa, Francisella philomiragia
  misc_feature (1)..(249): CP009442.1_translation Francisella philomiragia strain O#319-036 [FSC 153], NadE
MKIIKNFIAEEYSKKLIEWLKKICINYPAEGFVIGISGGIDSAVAASLAVKTGLPTTALILPSKNNQDQDMKDGLELIKNLDIEHHIVPIQPAYDTFIESTLNFTNSQNDRQHVIKGNAQARLRMMYLYAYAQQNNRIVIGTDNACEWYMGYFTKFGDGAADILPLVNLKKSQVFEMGEYLKVPQNIIDKAPSAGLWQGQTDEDEMGVTYQEIDNFLDGKEVSAKALERINFWHNRSHHKRSMAFTPNF

SEQ ID NO: 8 - protein, 249 aa, Francisella noatunensis
  misc_feature (1)..(249): CP003402.1_translation Francisella noatunensis subsp. orientalis str. Toba 04, NadE
MKIIKNFIAKEYSKKLIEWLKKICINYPAEGFVIGISGGIDSAVAASLAVKTGLPTTALILPSKNNQDQDMKDGLELIKNLDIEHHIVPIQPAYDTFIESTFNFTNAQNNRQHVIKGNAQARLRMMYLYAYAQQNNRIVIGTDNACEWYMGYFTKFGDGAADILPLVNLKKSQVFELGKYLKVPQNIIDKAPSAGLWQGQTDEDEMGVSYKEIDDFLDGKEVSEKALERINFWHNRSHHKRSIAFTPDF

SEQ ID NO: 9 - protein, 249 aa, Francisella philomiragia
  misc_feature (1)..(249): CP009440.1_translation Francisella philomiragia strain GA01-2794, NadE
MKIIKNFIVEKYSKKLIEWLKKICINYPAEGFVIGISGGIDSAVAASLAVKTGLPTTALILPSKNNQDQDMKDGLELIKNLDIEHHIVPIQPAYDTFIESTFNFTNAQNNRQHVIKGNAQARLRMMYLYAYAQQNNRIVIGTDNACEWYMGYFTKFGDGAADILPLVNLKKSQVFELGKYLKVPQNIIDKAPSAGLWQGQTDEDEMGVSYKEIDDFLDGKEVSEKALERINFWHNRSHHKRSIAFTPDF

SEQ ID NO: 10 - protein, 249 aa, Francisella persica
  misc_feature (1)..(249): CP013022.1_translation Francisella persica ATCC VR-331, NadE
MKIVKDFNIKEYSQKLIDWLSDTCMNYPAEGFVIGLSGGIDSAVAASLAVKTGLSTTALILPSKNNQHQDIQDALELADKINIEHHTITIQTVYETFLASIKKITNTERDRQLVIKGNAQARLRMMYLYAYAQQYNRVVIGTDNACEWYMGYFTKFGDGAADILPLVNLKKSHVFELGKYLGVPKNILDKAPSAGLWQGQTDEDEMGVTYQEIDDFLDGKQVSAKALERINFWHNRSHHKRKLALIPNF

SEQ ID NO: 11 - protein, 249 aa, Francisella cf. novicida
  misc_feature (1)..(249): CP002558.1_translation Francisella cf. novicida 3523, NadE
MKIVKDFSPKEYSQNLVNWLSDTCINYPAEGFVIGISGGIDSAVAASLAVKTGLPTTALILPSKNNQHQDIQDALELVEKLNIEHHIVTIQPAYENFLASTQEFINTDNNRQLVIKGNAQARLRMMYLYAYAQQYNRIVIGTDNACEWYMGYFTKFGDGAADIFPLINLKKSQVFELGKYLDVPKNIIDKAPSAGLWQGQTDEDEMGVTYQEIDDFLDGKQISAKALERINFWHNRSHHKRKLALTPNF

SEQ ID NO: 12 - protein, 249 aa, Francisella tularensis
  misc_feature (1)..(249): CP009607.1_translation Francisella tularensis subsp. novicida D9876, NadE
MKIVKDFSPKEYSQKLVNWLSDSCMNYPAEGFVIGLSGGIDSAVAASLAVKTGLPTTALILPSDNNQHQDMQDALDLIEMLNIEHYTISIQPAYEAFLASTQRFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 13 - protein, 249 aa, Francisella tularensis
  misc_feature (1)..(249): CP009353.1_translation Francisella tularensis subsp. novicida F6168, NadE
MKIVKDFSPKEYSQKLVNWLSDSCMNYPAEGFVIGLSGGIDSAVAASLAVKTGLPITALILPSDNNQHQDMQDALDLIEMLNIEHYTISIQPAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 14 - protein, 249 aa, Francisella tularensis
  misc_feature (1)..(249): CP010115.1_translation Francisella tularensis subsp. tularensis strain NIH B-38, NadE
MKIVKDFSPKEYSQKLVNWLSDSCMNYPAEGFVIGLSGGIDSAVAASLAVKTGLPTTALILPSDNNQHQDMQDALELIEMLNIEHYTISIQLAYEAFLASTQSFTNLQNNRQLVIKGNAQARLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALEIINFWHNRSHHKRKLALTPNF

SEQ ID NO: 15 - protein, 249 aa, Francisella tularensis
  misc_feature (1)..(249): CP003932.1_translation Francisella tularensis subsp. holarctica F92, NadE
MKIVKDFSPKEYSQKLVNWLSDSCMNYPAEGFVIGLSGGIDSAVAASLAVKTGLPTTALILPSDNNQHQDMQDALELIEMLNIEHYTISIQPAYEAFLASTQSFTNLQNNRQLVIKGNAQTRLRMMYLYAYAQQYNRIVIGTDNACEWYMGYFTKFGDGAADILPLVNLKKSQVFELGKYLDVPKNILDKAPSAGLWQGQTDEDEMGVTYQEIDDFLDGKQVSAKALERINFWHNRSHHKRKLALTPNF

SEQ ID NO: 16 - protein, 239 aa, Dichelobacter nodosus
  misc_feature (1)..(239): CP000513.1_translation Dichelobacter nodosus VCS1703A, NadE
QYIDYLLVWLEEQRAHLYASDGYTLGVSGGIDSAVCLHLLAKTGKPVQALVLPINANANDCEDAELVLKNANISGNIIALDDVYTAAQNTLAPVLNRDYERMPVLNGNLMARLRMVMLYTVAQSHRSVVVGTDNAVEYYLGYFTKFGDGACDILPLAKLTKSEVGQLAKALGVPKKIREKAPSAGLWQGQTDENEIGVSYADLDAFLCGKTVDDAVREKIAYWHQRSHHKRMLPPMPEI

SEQ ID NO: 17 - protein, 245 aa, Mannheimia succiniciproducens
  misc_feature (1)..(245): AE016827.1_translation Mannheimia succiniciproducens MBEL55E, NadE
KRMKTAAYADYLIQWLENQRTELYGMDGYTLGVSGGIDSAVCAHLAARTGAPVQALILPAEVTSPSDVADAQATLESAGIDGQIISIAPWYDLIMQQLSPVLNSEPERVNVLKGNLMARLRMIALFTTAQSHRSIVLGTDNAAEWLTGYFTKFGDGAADVLPLAGLRKEQVFELGRYLGVPQSVLDKKPSAGLWAGQTDEAEMGVTYAEIDAYLRGETVSPQALQQIRFWHNRSHHKRMLPPKPK

SEQ ID NO: 18 - protein, 238 aa, Actinobacillus succinogenes
  misc_feature (1)..(238): CP000746.1_translation Actinobacillus succinogenes 130Z, NadE
YVDYLVRWLETQRTELYGMDGYTLGVSGGIDSAVCAHLAARTGAPVQALILPAEVTSPEDVADAQITLESAGIDGRIISIAPWYDLIMLQLTPALNAESERINVLKGNLMARLRMIALFTTAQSHRSIVLGTDNAAEMLTGYFTKFGDGAADVLPLARLRKEQVFELGRYLGVPKSVLEKKPSAGLWAGQTDEGEMGVSYAEIDAYLRGETVSPQALKQIQFWHNRSHHKRMLPPTPE

SEQ ID NO: 19 - DNA, 753 bp, Mannheimia succiniciproducens
  misc_feature (1)..(753): AE016827 Mannheimia succiniciproducens MBEL55E, NadE
atgaaaacggcagcatacgcagattatttaattcaatggctggaaaaccaacgaaccgaactttacgggatggacggctatacactgggcgtcagcggcggtattgacagcgccgtctgcgctcatttggcagcgcgcaccggcgcaccggtacaagccttaattttgcccgccgaagtaaccagtccgtcagatgtggcggatgcgcaagccacactggaaagcgccggtattgacggacaaataatttccattgcaccttggtacgatttaattatgcaacaactttccccggtattaaattccgaaccggagcgcgttaacgtattaaagggtaatttaatggcaagactgcgtatgattgcgctgtttaccacggcacaaagccatcgttctattgtgttaggcaccgataatgcggcggaatggctgacgggttattttaccaaattcggcgacggcgcagcggacgtactgcctttagcgggattgcgcaaagagcaggtatttgaactcggacgttatcttggcgtaccgcaaagcgtgctggataaaaaaccgagcgccggtttatgggcaggacaaacggacgaagctgaaatgggtgttacttatgcggaaatcgacgcttatctgcgcggcgaaaccgttagcccgcaggcattgcaacaaatccgtttctggcacaaccgttctcatcacaaacgtatgttgccacctaaaccgaaatcacccgatgaagcggagtgttaa

SEQ ID NO: 20 - DNA, 747 bp, Dichelobacter nodosus
  misc_feature (1)..(747): CP000513 Dichelobacter nodosus VCS1703A, NadE
atgaccgttcatcaatacatcgattatttactcgtgtggttagaagagcagcgcgctcatctttatgcatcagatggttatacgttgggcgtcagcggcggcatcgattccgccgtttgtctgcatttactcgccaaaacgggaaaacccgtgcaagcgttagttttgccgatcaatgcgaacgcgaacgattgtgaagatgccgaattagtgttaaaaaatgctaatatttccggcaatattatcgcgctcgatgatgtttataccgccgcacaaaacaccttggcgcctgttttaaatcgcgattatgaacgtatgcccgtattaaacggcaatttaatggcgcggctgcgtatggttatgctttataccgtggcgcaaagtcatcgttcggtggtcgtgggaacggataacgcggtggaatattatttaggttactttacaaaatttggcgacggcgcctgcgatattttgccgctggcaaaactgacaaaatcagaagtaggacaattggcaaaagcgttaggcgttccgaaaaaaatccgagaaaaagcgccgagcgcaggcttgtggcaagggcaaaccgatgaaaacgaaatcggcgtatcgtacgcggatttagatgcttttttgtgcggtaaaaccgttgatgatgccgtcagagaaaaaattgcttattggcatcaacgctcgcatcataaaagaatgttgccgccgatgccggaaatcggattatctttggcgtaa

SEQ ID NO: 21 - DNA, 750 bp, Actinobacillus succinogenes
  misc_feature (1)..(750): CP000746 Actinobacillus succinogenes 130Z, NadE
atgagaacggcagcatacgtagattatttagtgcgatggctggaaacccagcgtaccgaattatacggtatggacggctacacgctgggggtcagcggcggtatcgacagtgccgtttgcgcccatttagcggcacgcaccggcgcccccgtacaggcattgattttacccgccgaagtcaccagccctgaagatgtggcggatgctcagattaccttggaaagtgcaggtattgacgggcggattatttctatcgctccttggtacgatttaattatgctacaacttacccccgcattaaatgcggaatctgaacgcattaacgtattgaaaggtaacttaatggcgcgcttacgtatgatcgcattatttaccacggcgcaaagccaccgttctatcgtattgggtacggataacgccgccgaaatgttaacgggctatttcaccaaattcggcgacggtgcggcggacgtattgccgttagcgaggttgcgcaaagaacaggtattcgaattagggcgttatcttggcgtaccgaaatccgtgctggagaaaaaaccgagtgcgggcttatgggcggggcaaacggacgagggggaaatgggtgtcagttatgcggaaatcgacgcctatctgcgcggcgaaaccgtcagtccgcaggcgttaaagcagattcaattctggcacaaccgttctcatcacaaacgtatgctgccgccgacgccagaaccgccggatgaaatcgattaa

SEQ ID NO: 22 - DNA, 750 bp, Francisella cf. novicida
  misc_feature (1)..(750): CP002558 Francisella cf. novicida 3523, NadE
atgaaaatagttaaagattttagtcctaaagaatattcacaaaatttagttaattggctaagtgatacttgtataaattatcctgctgaaggatttgtaatcggcattagcggtggtatagattcagctgttgcagcttctttagctgtcaaaactggattaccaactacagctttaatactaccttcaaaaaacaatcaacaccaagatatccaagatgctctagaacttgttgagaaacttaatattgaacatcatattgttacaattcaaccagcatacgaaaattttctagcatcaacacaggaatttataaatacagataataatagacaacttgtgatcaagggaaatgctcaagcacgtttaaggatgatgtatttatatgcctatgcccaacaatataacagaatagttataggtactgataatgcttgtgagtggtatatgggatattttacaaaatttggtgatggcgctgctgatatatttccgctaattaatcttaaaaaatcacaagtttttgaattaggtaaatacttagatgttccgaaaaatataattgataaagctccgtctgctggactatggcaaggacaaactgatgaggatgaaatgggcgtaacttatcaagaaattgatgatttcttagatggtaaacaaatttcagcaaaagccctagaaagaataaacttctggcataatcgtagtcatcataagagaaaactagctttaactcctaatttctaa

SEQ ID NO: 23 - DNA, 750 bp, Francisella sp
  misc_feature (1)..(750): CP002872 Francisella sp. TX077308, NadE
atgaaaatagtaaaaaactttattgtagaacagtattctaataatttaataaaatggttaaaagagaattgcataaaatatcctgctgaaggttttgtgattggtattagtggtggtatcgattcggcagtagccgcatctttagcagtcaaaacaggattacctacaactgctctaatattgccatcgaagaacaatcaagaccaagatatgcgagatggaatagaactaatcgaaaatcttaatatagagtatcatactgtttcaatacaacctgcttatgacacgtttatagagtcaacatttaactttacaaactcacaaaatgatcgccaacatgttatcaaaggaaatgcccaagcgcgtcttagaatgatgtatttatatgcttatgctcagcaaaataatagaattgttataggtacagataacgcatgtgaatggtacatgggatatttcactaaatttggtgatggtgcagcagatatattaccacttattaatctcaaaaaatctcaagtttttgaactaggtaaatacttaaaagtgccaaaaaacattatccaaaaagatccttctgccggtctatggcaaggtcaaactgatgaggatgaaatgggtgtcacatacaaagaaattgatgacttcttagacggtaaagaagtctcagaaaaagctctcgaaagaataagcttctggcataatcgtagtcaccataaaagatccatggcttttacccctaatttttaa

SEQ ID NO: 24 - DNA, 750 bp, Francisella sp.
  misc_feature (1)..(750): CP009574 Francisella sp. FSC1006, NadE
atgagtgtagtaaaaaattttaaacctaatgaatatgccaataaaattactgaatggctgaaagactcttgtttaaattatcccgctgaaggttttgtggtaggtattagtggaggtatagattcagcagtagcagtctctttagcagtaaatactggactacctgttacagggctaataatgccatcaaaaaataatgatgataaagataccttagatgctatagaattagctaaaaaattaaatatagaatatcatctcatacccattcaaccagtatatgaaacatttctagattcagctgaagatatcaaaaacagtgctaatgaccgtcaacatgtaatcaaaggaaatgcacaagctcgttttagaatgatatacttgtatgcttacgctcagcaaaataatagaatggtaattggtacagataatgcttgtgaatggtatatgggctattttacaaaatttggagatggagccgctgatatactgcctcttataaaattaaaaaaatcacaagtttttgaattaggtagctatcttaatgtacccaataacatcctcacaaaagctccttccgcaggactttggcttggacaaactgatgaagcagagatgggggtttcatatcaagaaatagatgatttccttgatggtaaacatgtctcagattatgctcttaatcaaataaaattctggcataaccgtagtcatcataaaagaatcatggctaaggctccagatttttaa

SEQ ID NO: 25 - DNA, 753 bp, Francisella guangzhouensis
  misc_feature (1)..(753): CP010427 Francisella guangzhouensis strain 08HL01032, NadE
atgaacgtagtaaaaaatttcactcctgaaaaatattcagaaaaacttatacaatggctcactaatagctgtataaaatatcctgcagaaggtttcgtaattggtgtaagtggtggtatagattctgcagtatgtgcatcacttttatccaaaactgatcttcctacaacagcttttatactaccatcaaaaaataactctgatcaagatatgatcgatgcattagaacttataaataaattaaatattccataccatataataccaatccagccagtttatgaaagttttctaaagtccacacagctatttacaaatccacaaaatgacagacaaaatgtcataaaaggtaacgctcaagctcgttttagaatgatgtatttatatgcttatgcacaacaaaataatcgtatagtagttggaacagataatgcttgtgaatggtatatgggttatttcaccaaatttggcgatggagctgctgatatactaccattaataaatcttaaaaagtcccaggtatttgagttaggtaaatacttagatgttccaaggaatatcctaactaaggcaccctctgctggtctttggcaaggccaaactgatgaaggtgaaatgggagttacttatcaggaaatagataattttctcgacggtaaagaagtatcgccagcaacttttgaaaaaataagctactggcataatcgctctcaccacaaaagaaagatggctttaacgccagattttaactaa

SEQ ID NO: 26 - DNA, 750 bp, Francisella persica
  misc_feature (1)..(750): CP013022 Francisella persica ATCC VR-331, NadE
atgaaaatagttaaagatttcaacatcaaagaatattcacaaaagttaattgattggctaagtgatacttgtatgaattaccctgctgaaggatttgtcattggtcttagcggtggtatagattcggcagttgcagcttctttagctgtcaaaactggattatcaactacagctttaatattaccatcaaaaaacaatcaacaccaagatatacaagatgctctagaacttgcagataaaattaatattgaacatcatactattacaattcaaacagtatacgaaacttttcttgcgtcaataaaaaaaattacaaataccgaacgtgatagacaacttgtcattaaaggaaatgctcaagctcgtttgaggatgatgtatttatatgcctatgctcaacaatataatagagtggttattggtactgataatgcttgtgaatggtatatgggatattttacaaagtttggtgatggtgctgctgatattcttccactagttaatctcaaaaaatctcacgtttttgaattaggtaaatacttaggtgttcctaaaaatatacttgataaagctccatctgctgggctatggcaaggacaaactgatgaagatgaaatgggcgtaacttatcaagaaattgatgatttcttagatggtaagcaagtttcagcgaaagctctagaaagaataaatttctggcataatcgtagtcatcataagagaaaactagctttaattcctaatttctaa

SEQ ID NO: 27 - DNA, 753 bp, Mannheimia succiniciproducens
  misc_feature (1)..(753): Optimized_AE016827 Mannheimia succiniciproducens MBEL55E, NadE
atgaaaacggcagcatacgcagattatctgattcaatggctggaaaaccaacgcaccgaactgtacggcatggacggctataccctgggcgtcagcggcggtattgacagcgccgtctgcgctcatctggcagcgcgcaccggcgcgccggtacaagccctgattctgccggcggaagtaaccagtccgtcagatgtggcggatgcgcaagccaccctggaaagcgccggtattgacggccaaattatttccattgcaccgtggtacgatctgattatgcaacaactgtccccggtactgaatagcgaaccggagcgcgttaacgtactgaaaggtaatctgatggcacgcctgcgtatgattgcgctgtttaccacggcacaaagccatcgttctattgtgctgggcaccgataatgcggcggaatggctgacgggttattttaccaaattcggcgacggcgcagcggacgtactgccgctggcgggcctgcgcaaagagcaggtatttgaactgggccgttatctgggcgtaccgcaaagcgtgctggataaaaaaccgagcgccggtctgtgggcaggccaaacggacgaagctgaaatgggtgttacctatgcggaaatcgacgcttatctgcgcggcgaaaccgttagcccgcaggcactgcaacaaatccgtttctggcacaaccgttctcatcacaaacgtatgctgccgccgaaaccgaaatcaccggatgaagcggagtgttaa

SEQ ID NO: 28 - DNA, 747 bp, Dichelobacter nodosus
  misc_feature (1)..(747): Optimized_CP000513 Dichelobacter nodosus VCS1703A, NadE
atgaccgttcatcaatacattgattatctgctggtgtggctggaagagcagcgcgctcatctgtatgcatcagatggttatacgctgggcgtcagcggcggcattgattccgccgtttgtctgcatctgctggccaaaacgggcaaaccagtgcaagcgctggttctgccgatcaatgcgaacgcgaacgattgtgaagatgccgaactggtgctgaaaaatgctaatatttccggcaatattatcgcgctggatgatgtttataccgccgcacaaaacaccctggcgccggttctgaatcgcgattatgaacgtatgccggtactgaacggcaatctgatggcgcgcctgcgtatggttatgctgtataccgtggcgcaaagtcatcgttcggtggtcgtgggcacggataacgcggtggaatattatctgggttactttaccaaatttggcgacggcgcctgcgatattctgccgctggcaaaactgaccaaatcagaagtaggccaactggcaaaagcgctgggcgttccgaaaaaaatccgcgaaaaagcgccgagcgcgggcctgtggcaaggccaaaccgatgaaaacgaaatcggcgtatcgtacgcggatctggatgcttttctgtgcggtaaaaccgttgatgatgccgtccgcgaaaaaattgcttattggcatcaacgctcgcatcataaacgtatgctgccgccgatgccggaaatcggcctgtctctggcgtaa

SEQ ID NO: 29 - DNA, 750 bp, Actinobacillus succinogenes
  misc_feature (1)..(750): Optimized_CP000746 Actinobacillus succinogenes 130Z, NadE
atgcgcacggcagcatacgtagattatctggtgcgctggctggaaacccagcgtaccgaactgtacggtatggacggctacacgctgggcgtcagcggcggtatcgacagtgccgtttgcgcccatctggcggcacgcaccggcgccccggtacaggcactgattctgccggcggaagtcaccagcccggaagatgtggcggatgctcagattaccctggaaagtgcaggtattgatggccgcattatttctatcgctccgtggtacgatctgattatgctgcaactgaccccggcactgaatgcggaatctgaacgcattaacgtactgaaaggtaacctgatggcgcgcctgcgtatgatcgcactgtttaccacggcgcaaagccaccgttctatcgtactgggtacggataacgccgccgaaatgctgacgggctatttcaccaaattcggcgacggtgcggcggacgtactgccgctggcgcgcctgcgcaaagaacaggtattcgaactgggccgttatctgggcgtaccgaaatccgtgctggagaaaaaaccgagtgcgggcctgtgggcgggccaaacggacgagggcgaaatgggtgtcagttatgcggaaatcgacgcctatctgcgcggcgaaaccgtcagtccgcaggcgctgaaacagattcaattctggcacaaccgttctcatcacaaacgtatgctgccgccgacgccggaaccgccggatgaaattgattaa

SEQ ID NO: 30 - DNA, 750 bp, Francisella cf. novicida
  misc_feature (1)..(750): Optimized_CP002558 Francisella cf. novicida 3523, NadE
atgaaaattgttaaagattttagtccgaaagaatattcacaaaatctggttaattggctgagtgatacctgtattaattatccggctgaaggctttgtaatcggcattagcggtggtattgattcagcggttgcagcttctctggctgtcaaaaccggcctgccgaccaccgctctgattctgccgtcaaaaaacaatcaacaccaagatattcaagatgctctggaactggttgagaaactgaatattgaacatcatattgttaccattcaaccggcatacgaaaattttctggcatcaacccaggaatttattaataccgataataatcgccaactggtgatcaaaggcaatgctcaagcacgtctgcgcatgatgtatctgtatgcctatgcccaacaatataaccgcattgttattggtaccgataatgcttgtgagtggtatatgggctattttaccaaatttggtgatggcgctgctgatatttttccgctgattaatctgaaaaaatcacaagtttttgaactgggtaaatacctggatgttccgaaaaatattattgataaagctccgtctgctggcctgtggcaaggccaaaccgatgaggatgaaatgggcgtaacctatcaagaaattgatgatttcctggatggtaaacaaatttcagcaaaagccctggaacgcattaacttctggcataatcgtagtcatcataaacgcaaactggctctgaccccgaatttctaa

SEQ ID NO: 31 - DNA, 750 bp, Francisella sp.
  misc_feature (1)..(750): Optimized_CP002872 Francisella sp. TX077308, NadE
atgaaaattgtaaaaaactttattgtagaacagtattctaataatctgattaaatggctgaaagagaattgcattaaatatccggctgaaggttttgtgattggtattagtggtggtattgattcggcagtagccgcatctctggcagtcaaaaccggcctgccgaccaccgctctgattctgccgtcgaaaaacaatcaagaccaagatatgcgcgatggcattgaactgatcgaaaatctgaatattgagtatcataccgtttcaattcaaccggcttatgacacgtttattgagtcaacctttaactttaccaactcacaaaatgatcgccaacatgttatcaaaggcaatgcccaagcgcgtctgcgcatgatgtatctgtatgcttatgctcagcaaaataatcgcattgttattggtaccgataacgcatgtgaatggtacatgggctatttcaccaaatttggtgatggtgcagcagatattctgccgctgattaatctgaaaaaatctcaagtttttgaactgggtaaatacctgaaagtgccgaaaaacattatccaaaaagatccgtctgccggtctgtggcaaggtcaaaccgatgaggatgaaatgggtgtcacctacaaagaaattgatgacttcctggacggtaaagaagtctcagaaaaagctctggaacgcattagcttctggcataatcgtagtcaccataaacgcagcatggcttttaccccgaatttttaa

SEQ ID NO: 32 - DNA, 750 bp, Francisella sp.
  misc_feature (1)..(750): Optimized_CP009574 Francisella sp. FSC1006, NadE
atgagtgtagtaaaaaattttaaaccgaatgaatatgccaataaaattaccgaatggctgaaagactcttgtctgaattatccggctgaaggttttgtggtaggtattagtggcggtattgattcagcagtagcagtctctctggcagtaaataccggcctgccggttaccggcctgattatgccgtcaaaaaataatgatgataaagataccctggatgctattgaactggctaaaaaactgaatattgaatatcatctgattccgattcagccggtatatgaaacctttctggattcagcggaagatattaaaaacagtgctaatgaccgtcaacatgtaatcaaaggcaatgcacaagctcgttttcgcatgatttacctgtatgcttacgctcagcaaaataatcgcatggtaattggtaccgataatgcttgtgaatggtatatgggctattttaccaaatttggcgatggcgccgctgatattctgccgctgattaaactgaaaaaatcacaagtttttgaactgggtagctatctgaatgtaccgaataacatcctgaccaaagctccgagcgcgggcctgtggctgggccaaaccgatgaagcagagatgggcgtttcatatcaagaaattgatgatttcctggatggtaaacatgtctcagattatgctctgaatcaaattaaattctggcataaccgtagtcatcataaacgcatcatggctaaagctccggatttttaa

SEQ ID NO: 33 - DNA, 753 bp, Francisella guangzhouensis
  misc_feature (1)..(753): Optimized_CP010427 Francisella guangzhouensis strain 08HL01032, NadE
atgaacgtagtaaaaaatttcaccccggaaaaatattcagaaaaactgattcaatggctgaccaatagctgtattaaatatccggcagaaggtttcgtaattggtgtaagtggtggtattgattctgcggtatgtgcatcactgctgtccaaaaccgatctgccgaccaccgcttttattctgccgtcaaaaaataactctgatcaagatatgattgatgcactggaactgattaataaactgaatattccgtaccatattattccgatccagccggtttatgaaagttttctgaaatccacccaactgtttaccaatccgcaaaatgaccgccaaaatgtcattaaaggtaacgctcaagctcgttttcgcatgatgtatctgtatgcttatgcacaacaaaataatcgtattgtagttggcaccgataatgcttgtgaatggtatatgggttatttcaccaaatttggcgatggcgctgctgatattctgccgctgattaatctgaaaaaatcccaggtatttgagctgggtaaatacctggatgttccgcgcaatatcctgaccaaagcaccgtctgctggtctgtggcaaggccaaaccgatgaaggtgaaatgggcgttacctatcaggaaattgataattttctggacggtaaagaagtatcgccggcaacctttgaaaaaattagctactggcataatcgctctcaccacaaacgcaaaatggctctgacgccggattttaactaa

SEQ ID NO: 34 - DNA, 750 bp, Francisella persica
  misc_feature (1)..(750): Optimized_CP013022 Francisella persica ATCC VR-331, NadE
atgaaaattgttaaagatttcaacatcaaagaatattcacaaaaactgattgattggctgagtgatacctgtatgaattacccggctgaaggctttgtcattggtctgagcggtggtattgattcggcagttgcagcttctctggctgtcaaaaccggcctgtcaaccaccgctctgattctgccgtcaaaaaacaatcaacaccaagatattcaagatgctctggaactggcagataaaattaatattgaacatcataccattaccattcaaaccgtatacgaaacctttctggcgtcaattaaaaaaattaccaataccgaacgtgatcgccaactggtcattaaaggcaatgctcaagctcgtctgcgcatgatgtatctgtatgcctatgctcaacaatataatcgcgtggttattggtaccgataatgcttgtgaatggtatatgggctattttaccaaatttggtgatggtgctgctgatattctgccgctggttaatctgaaaaaatctcacgtttttgaactgggtaaatacctgggtgttccgaaaaatattctggataaagctccgtctgctggcctgtggcaaggccaaaccgatgaagatgaaatgggcgtaacctatcaagaaattgatgatttcctggatggtaaacaagtttcagcgaaagctctggaacgcattaatttctggcataatcgtagtcatcataaacgcaaactggctctgattccgaatttctaa

SEQ ID NO: 35 - DNA, 750 bp, Unknown
  FtNadE_star_optimized
  misc_feature (1)..(750): FtNadE_star_optimized
atgaaaatcgtcaaagacttctccccgaaagaatattcccaaaaactggtgaactggctgagcgactcgtgtatgaactatccggcagaaggctttgtcattggtctgagtggcggtatcgattccgctgtggcggcctcactggccgttaaaaccggcctgccgaccacggcactgattctgccgtctgacaacaatcagcatcaagatatgcaggacgcgctggaactgattgaaatgctgaacatcgaacactacaccatttccatccagccggcgtatgaagcgtttctggcgagcacccaatctttcacgaacctgcagaacaatcgtcaactggtgatcaaaggcaatgcgcaggcccgtctgcgcatgatgtatctgtacgcgtatgcgcagcaatacaaccgcattgttatcggcaccgataatgcctgcgaatggtacatgggttattttacgaaattcggcgatggtgcagctgacattctgccgctggtcaacctgaaaaaatcgcaggtgtttgaactgggtaaatacctggatgttccgaaaaatatcctggacaaagcaccgagcgcaggtctgtggcagggtcaaaccgatgaagacgaaatgggcgttacgtatcaggaaattgatgacttcctggatggtaaacaagtcagcgcgaaagccctggaacgtatcaacttctggcacaaccgctcacatcataaacgcaaactggcactgaccccgaacttctaa

SEQ ID NO: 36 - DNA, 750 bp, Unknown
  DQ682092-AL_Reverted
  misc_feature (1)..(750): DQ682092-AL_Reverted
atgaaaatcgtcaaagacttctccccgaaagaatattcccaaaaactggtgaactggctgagcgactcgtgtatgaacactccggcagaaggctttgtcattggtctgagtggcggtatcgattccgctgtggcggcctcactggccgttaaaaccggcctgccgaccacggcactgattctgccgtctgacaacaatcagcatcaagatatgcaggacgcgctggaactgattgaaatgctgaacatcgaacactacaccatttccatccagccggcgtatgaagcgtttctggcgagcacccaatctttcacgaacctgcagaacaatcgtcaactggtgatcaaaggcaatgcgcaggcccgtctgcgcatgatgtatctgtacgcgtatgcgggtcaatacaaccgcattgttatcggcaccgataatgcctgcgaatggtacatgggttattttacgaaattcggcgatggtgcagctgacattctgccgctggtcaacctgaaaaaatcgcaggtgtttgaactgggtaaatacctggatgttccgaaaaatatcctggacaaagcaccgagcgcaggtctgtggcagggtcaaaccgatgaagacgaaatgggcgttacgtatcaggaaattgatgacttcctggatggtaaacaagtcagcgcgaaagccctggaacgtatcaacttctggcacaacgtctcacatcataaacgcaaactggcactgaccccgaacttctaa

SEQ ID NO: 37 - DNA, 753 bp, Mannheimia succiniciproducens
  misc_feature (1)..(753): Optimized_AE016827_Reverted Mannheimia succiniciproducens
atgaaaacggcagcatacgcagattatctgattcaatggctggaaaaccaacgcaccgaactgaccggcatggacggctataccctgggcgtcagcggcggtattgacagcgccgtctgcgctcatctggcagcgcgcaccggcgcgccggtacaagccctgattctgccggcggaagtaaccagtccgtcagatgtggcggatgcgcaagccaccctggaaagcgccggtattgacggccaaattatttccattgcaccgtggtacgatctgattatgcaacaactgtccccggtactgaatagcgaaccggagcgcgttaacgtactgaaaggtaatctgatggcacgcctgcgtatgattgcgctgtttaccacggcaggcagccatcgttctattgtgctgggcaccgataatgcggcggaatggctgacgggttattttaccaaattcggcgacggcgcagcggacgtactgccgctggcgggcctgcgcaaagagcaggtatttgaactgggccgttatctgggcgtaccgcaaagcgtgctggataaaaaaccgagcgccggtctgtgggcaggccaaacggacgaagctgaaatgggtgttacctatgcggaaatcgacgcttatctgcgcggcgaaaccgttagcccgcaggcactgcaacaaatccgtttctggcacaacgtttctcatcacaaacgtatgctgccgccgaaaccgaaatcaccggatgaagcggagtgttaa

SEQ ID NO: 38 - DNA, 750 bp, Francisella cf. novicida
  misc_feature (1)..(750): Optimized_CP002558_Reverted Francisella cf. novicida 3523
atgaaaattgttaaagattttagtccgaaagaatattcacaaaatctggttaattggctgagtgatacctgtattaatactccggctgaaggctttgtaatcggcattagcggtggtattgattcagcggttgcagcttctctggctgtcaaaaccggcctgccgaccaccgctctgattctgccgtcaaaaaacaatcaacaccaagatattcaagatgctctggaactggttgagaaactgaatattgaacatcatattgttaccattcaaccggcatacgaaaattttctggcatcaacccaggaatttattaataccgataataatcgccaactggtgatcaaaggcaatgctcaagcacgtctgcgcatgatgtatctgtatgcctatgccggccaatataaccgcattgttattggtaccgataatgcttgtgagtggtatatgggctattttaccaaatttggtgatggcgctgctgatatttttccgctgattaatctgaaaaaatcacaagtttttgaactgggtaaatacctggatgttccgaaaaatattattgataaagctccgtctgctggcctgtggcaaggccaaaccgatgaggatgaaatgggcgtaacctatcaagaaattgatgatttcctggatggtaaacaaatttcagcaaaagccctggaacgcattaacttctggcataatgttagtcatcataaacgcaaactggctctgaccccgaatttctaa

SEQ ID NO: 39 - DNA, 750 bp, Francisella sp.
  misc_feature (1)..(750): Optimized_CP002872_Reverted Francisella sp. TX077308
atgaaaattgtaaaaaactttattgtagaacagtattctaataatctgattaaatggctgaaagagaattgcattaaaactccggctgaaggttttgtgattggtattagtggtggtattgattcggcagtagccgcatctctggcagtcaaaaccggcctgccgaccaccgctctgattctgccgtcgaaaaacaatcaagaccaagatatgcgcgatggcattgaactgatcgaaaatctgaatattgagtatcataccgtttcaattcaaccggcttatgacacgtttattgagtcaacctttaactttaccaactcacaaaatgatcgccaacatgttatcaaaggcaatgcccaagcgcgtctgcgcatgatgtatctgtatgcttatgctgggcaaaataatcgcattgttattggtaccgataacgcatgtgaatggtacatgggctatttcaccaaatttggtgatggtgcagcagatattctgccgctgattaatctgaaaaaatctcaagtttttgaactgggtaaatacctgaaagtgccgaaaaacattatccaaaaagatccgtctgccggtctgtggcaaggtcaaaccgatgaggatgaaatgggtgtcacctacaaagaaattgatgacttcctggacggtaaagaagtctcagaaaaagctctggaacgcattagcttctggcataatgttagtcaccataaacgcagcatggcttttaccccgaatttttaa

SEQ ID NO: 40 - DNA, 828 bp, E. coli
  misc_feature (1)..(828): E. coli NadE
atgacattgcaacaacaaataataaaggcgctgggcgcaaaaccgcagattaatgctgaagaggaaattcgtcgtagtgtcgattttctgaaaagctacctgcaaacttatccgttcattaaatcactggtgctcgggatcagcggcggtcaggactccacgcttgccggaaagctgtgccagatggcgattaatgagctgcgcctggaaaccggcaacgaatcactgcaatttattgccgtacgcctgccctatggtgttcaggccgacgaacaagattgccaggatgccattgcctttattcaaccggatcgcgtattaaccgttaatatcaagggcgcggtattggccagcgaacaggcattgcgggaagcaggcattgaactgagcgattttgtccgtggcaatgaaaaagcgcgtgagcggatgaaagcacaatatagcattgcgggtatgaccagcggtgtcgtggtgggcaccgatcatgcagcagaagccattaccggattcttcactaaatatggtgacggcggtacggacattaatccgctgtatcgtctcaacaaacgtcagggtaaacagttactggcggcattagcttgcccggaacacctttataagaaagcgccaacggccgatctggaagatgatcgcccttctctgccagatgaagtggcactcggcgtgacctatgacaatatcgacgactatctggaagggaaaaacgtacctcaacaggtcgccagaacaatagagaactggtatctgaaaaccgaacataaacgccgtccgccaattaccgttttcgatgatttctggaaaaagtaa

SEQ ID NO: 41 - protein, 540 aa, Escherichia coli
  misc_feature (1)..(540): Escherichia coli NadB >gi|260448346|gb|ACX38768.1| L-aspartate oxidase [Escherichia coli DH1]
MNTLPEHSCDVLIIGSGAAGLSLALRLADQHQVIVLSKGPVTEGSTFYAQGGIAAVFDETDSIDSHVEDTLIAGAGICDRHAVEFVASNARSCVQWLIDQGVLFDTHIQPNGEESYHLTREGGHSHRRILHAADATGREVETTLVSKALNHPNIRVLERSNAVDLIVSDKIGLPGTRRVVGAWVWNRNKETVETCHAKAVVLATGGASKVYQYTTNPDISSGDGIAMAWRAGCRVANLEFNQFHPTALYHPQARNFLLTEALRGEGAYLKRPDGTRFMPDFDERGELAPRDIVARAIDHEMKRLGADCMFLDISHKPADFIRQHFPMIYEKLLGLGIDLTQEPVPIVPAAHYTCGGVMVDDHGRTDVEGLYAIGEVSYTGLHGANRMASNSLLECLVYGWSAAEDITRRMPYAHDISTLPPWDESRVENPDERVVIQHNWHELRLFMWDYVGIVRTTKRLERALRRITMLQQEIDEYYAHFRVSNNLLELRNLVQVAELIVRCAMMRKESRGLHFTLDYPELLTHSGPSILSPGNHYINR

SEQ ID NO: 42 - protein, 531 aa, Bacillus subtilis
  misc_feature (1)..(531): Bacillus subtilis NadB >gi|16079839|ref|NP_ 390665.1| L-aspartate oxidase [Bacillus subtilis subsp. subtilis str. 168]
MSKKTIAVIGSGAAALSLAAAFPPSYEVTVITKKSVKNSNSVYAQGGIAAAYAKDDSIEAHLEDTLYAGCGHNNLAIVADVLHDGKMMVQSLLERGFPFDRNERGGVCLGREGAHSYNRIFHAGGDATGRLLIDYLLKRINSKIKLIENETAADLLIEDGRCIGVMTKDSKGRLKVRHADEVVLAAGGCGNLFLHHTNDLTVTGDGLSLAYRAGAELTDLEFTQFHPTLLVKNGVSYGLVSEAVRGEGGCLVDENGRRIMAERHPLGDLAPRDIVSRVIHEEMAKGNRVYIDFSAISDFETRFPTITAICEKAGIDIHSGKIPVAPGMHFLMGGVSVNRWGETTVPGLYAIGETACSGLHGANRLASNSLLEALVFGKRAAEHIIQKPVYNRQYQSGLETSVFYEVPDIEGHELQSKMTSHMSILREQSSLIELSIWLHTLPFQEVNVKDITIRQMELSHLWQTAKLMTFSALLREESRGAHFRTDFPHAEVSWQGRQIVHTKKGTKIRKNEGIWNNESFTAEKITESLFS

SEQ ID NO: 43 - protein, 280 aa, Unknown
  NMN nucleotidase
  misc_feature (1)..(280): Sc SDT1 - NMN nucleotidase
MTVEYTASDLATYQNEVNEQIAKNKAHLESLTHPGSKVTFPIDQDISATPQNPNLKVFFFDIDNCLYKSSTRIHDLMQQSILRFFQTHLKLSPEDAHVLNNSYYKEYGLAIRGLVMFHKVNALEYNRLVDDSLPLQDILKPDIPLRNMLLRLRQSGKIDKLWLFTNAYKNHAIRCLRLLGIADLFDGLTYCDYSRTDTLVCKPHVKAFEKAMKESGLARYENAYFIDDSGKNIETGIKLGMKTCIHLVENEVNEILGQTPEGAIVISDILELPHVVSDLF

SEQ ID NO: 44 - protein, 450 aa, Unknown
  NMN nucleotidase
  misc_feature (1)..(450): Sc ISN1 - NMN nucleotidase
MSSRYRVEYHLKSHRKDEFIDWVKGLLASPFVLHAVSHEGDYNDDLATTQRVRSQYADIFKDIEGLIKDKIEFDSRNMSQDEIEDGASSQSLNILGQSRLNLLVPSIGTFFTELPLEQAFLWEDSQRAISARRMVAPSFNDIRHILNTAQIFHFKKQENLHNGKVLRLVTFDGDVTLYEDGGSLVYTNPVIPYILKLLRCGINVGIVTAAGYDEAGTYENRLKGLIVALHDSTDIPVSQKQNLTIMGGESSYLFRYYEDPEEDNFGFRQIDKEEWLLPRMKAWSLEDVEKTLDFAERTLNRLRKRLNLPSEISIIRKVRAVGIVPGERYDEASKRQVPVKLDREQLEEIVLTLQNTLESFAPSRRIQFSCFDGGSDVWCDIGGKDLGVRSLQQFYNPESPIQPSETLHVGDQFAPVGSANDFKARLAGCTLWIASPQETVNYLHRLLETD

SEQ ID NO: 45 - protein, 283 aa, Unknown
  NMN nucleotidase
  misc_feature (1)..(283): CAG78734 - NMN nucleotidase
MTQDTPKAEGIVLFVDIDNTLYSKDKGVDALMGERINNYFQTKLGLDKEHATELHHRYYKEYGIALDGLLRHHNIDIDEYNRLVDDSLPLDKILKRDEPLRQMFQRLDRTKVSKLWLFTNAYKTHGERVAKLVGVDDLIDGLTYCDYYHTPLHCKPKPEAFEKAMKQAGVTDKSKCWFVDDSALNIKASKKFGWAENAYLHYPGAPEIPAGTPGVVEISHIEDLPKVWPDLFLPEPVKNTNGTINGTTNGTTNGTTNGIINGTNGGINGDGTNGSTNGGNPPS

SEQ ID NO: 46 - protein, 420 aa, Unknown
  NMN nucleotidase
  misc_feature (1)..(420): CAG79432 - NMN nucleotidase
MTSRYRVEYSLKQHRRDEFIEWVKTLLATPFVLHAGSTGDGRPGDENRSVVETRRRYAEIFYDVEKLIENQLYHDNNDSGERARLRQLVPTIGQFFTKLPLERAFYTQDEIRSISHRRFVSPSFNDVRMILNTAQVMALATGRQPLKLVTFDGDVTLYADGGNIDPDSPIIPVLLGLLRNDVYVGIVTAAGYSEKDGSKYGVRLYGLLEAIVKTDTLTEQQKNHLLVMGGESNFLFKFIAAENRFEWIDPEEWTLPEVRTWSQEDITSVLNIGERVFTNALTKCSLPATIIRKVRGVGIVPLPGKKIMREQLEEMVLAAQKTLETSKATGNVQFCAFNGGSDVWVDIGDKDLGVRSLQAYLGNITGTQTLHVGDQFAALGANDFKARLAATTVWIANPSETVEIIAELIDYIEHERQCSA

SEQ ID NO: 47 - protein, 353 aa, Unknown
  NMN nucleotidase
  misc_feature (1)..(353): CAG82350 - NMN nucleotidase
MKLSSLIVPCLASLALAQTNATAHNSTVPHENSKTTIVVTNDDSWASANIRAFYDALKKEGYNVFMFAPAVQQSGTGGTFNLPTNATLAKGAEWQTAPAGAPSWAHDEKDDHIWYFDGTPGAAVSFGFDYALPKFYPNTTVDLVVSGPNEGWNLGPFVYTLSGTEGAMYTSILRGVPAVAFSGNNDHTYYANASNSETSAHKIYAKASTAIVKNLLENAKGRPSVLPIGVGISVNLPNVGDIDPTGKCVDPKPIFTRQTGRGAATYKLIFNETSGVFDEDENLKTDALRACFNGDCFLPDETDVVTKWGCYSSISVITADYDAPGGVAAEVQYLNRNLVTFAPTGYGSFPGRK

SEQ ID NO: 48 - protein, 349 aa, Unknown
  NMN nucleotidase
  misc_feature (1)..(349): CAG83794 - NMN nucleotidase
MKFSNLALPLLASCAVAQNSTNPNSNTTIIVTNDDSWASANIRSLYSELKKEGYNVFMFAPAVQQSGTGGTFNLPRAANLTKGGEFGSIPVGAPNWGQDDNDDHIWYFDGTPAAAMSFGLDYAIPRLFNNATVDLVVSGPNEGWNIGPNIYTMSGTNGAMYMAVLRGIPAIAYSGMNSHQYYANASTSDNAAHNIYAKAATGIVNNLVKNAKDRATLLPYGVGLSVNLPRAGDVDPTGQCKEIKPIFTRQTGPAAIVLKLSYNETTNRFSPGITNSEASKACLNGDCILPDESDVVANWGCYASVSVITTDYDAPRDVAGEVQFLNRGEVKFAPRVYGSFAPVEASPYF

SEQ ID NO: 49 - protein, 271 aa, Unknown
  NMN nucleotidase
  misc_feature (1)..(271): An02g02380 - NMN nucleotidase (SDT1 homolog)
MTTTSSPASAPLDPRPVFFFDIDNCLYSKGCNIHDEMQKLINQFFIKHLSLNADDAHMLHMKYYKEYGLAIEGLTRHHKIDPLEFNREVDDALPLDDILKPDPKLRRLLEDIDRSKVRMWLLTNAYVTHAKRVVKLLQVDDLFEGITYCDYGNSPLVCKPSQAMYERAEKEAGASSTSECYFVGKSPSQFLSHSTYPDQVLTDRSRRLRPELYSCCRPGLGCGSSCRAWNTPPPRAGLAIYDTKPGGTQDLFPHLIQDEAGSIDSITLCDR

SEQ ID NO: 50 - protein, 411 aa, Unknown
  NMN nucleotidase
  misc_feature (1)..(411): An18g01690 - NMN nucleotidase (ISN1 homolog)
MTTRYRVEYALKSHRRDQLIEWIKGLLAVPFVLHSQPTAVYQEHSENLIAVAADTHQRYAEIFRDVEMLIRDHITHQQDDAPGKSKLKLLVPTVGTFFTPLYLVDAFRRQDAQRFISRRRFVAPSFNDIRLILNSAQLIGLARTTGVDLVTFDGDVTLYDDGACLTDDNPVILRIMRLLLQGRKVGIVTAAGYTDAAKYYERLKGLLDAMYESAEMTDAQRAGLVVMGGESNFLFRYDHASPSRLSYVPREEWLLEEMKTWQEGDITRLLDIAESSLRACASNLNLPVAVLRKDRAVGVYPTERGRISREQLEETVLVVQNTVERSEVGARLPFCAFNGGNDVFVDIGDKSWGVRACQRYFGGIEPARTLHVGDQFLSAGANDFKARLASTTAWIASPAETVQLLDELQGI

SEQ ID NO: 51 - protein, 401 aa, Unknown
  NAMN adenyltransferase
  misc_feature (1)..(401): Sc NMA1 - NAMN adenyltransferase
MDPTRAPDFKPPSADEELIPPPDPESKIPKSIPIIPYVLADANSSIDAPFNIKRKKKHPKHHHHHHHSRKEGNDKKHQHIPLNQDDFQPLSAEVSSEDDDADFRSKERYGSDSTTESETRGVQKYQIADLEEVPHGIVRQARTLEDYEFPSHRLSKKLLDPNKLPLVIVACGSFSPITYLHLRMFEMALDAISEQTRFEVIGGYYSPVSDNYQKQGLAPSYHRVRMCELACERTSSWLMVDAWESLQPSYTRTAKVLDHFNHEINIKRGGVATVTGEKIGVKIMLLAGGDLIESMGEPNVWADADLHHILGNYGCLIVERTGSDVRSFLLSHDIMYEHRRNILIIKQLIYNDISSTKVRLFIRRAMSVQYLLPNSVIRYIQEHRLYVDQTEPVKQVLGNKE

SEQ ID NO: 52 - protein, 395 aa, Unknown
  NAMN adenyltransferase
  misc_feature (1)..(395): Sc NMA2 - NAMN adenyltransferase
MDPTKAPDFKPPQPNEELQPPPDPTHTIPKSGPIVPYVLADYNSSIDAPFNLDIYKTLSSRKKNANSSNRMDHIPLNTSDFQPLSRDVSSEEESEGQSNGIDATLQDVTMTGNLGVLKSQIADLEEVPHTIVRQARTIEDYEFPVHRLTKKLQDPEKLPLIIVACGSFSPITYLHLRMFEMALDDINEQTRFEVVGGYFSPVSDNYQKRGLAPAYHRVRMCELACERTSSWLMVDAWESLQSSYTRTAKVLDHFNHEINIKRGGIMTVDGEKMGVKIMLLAGGDLIESMGEPHVWADSDLHHILGNYGCLIVERTGSDVRSFLLSHDIMYEHRRNILIIKQLIYNDISSTKVRLFIRRGMSVQYLLPNSVIRYIQEYNLYINQSEPVKQVLDSKE

SEQ ID NO: 53 - protein, 258 aa, Unknown
  NMN adenyltransferase
  misc_feature (1)..(258): Sc POF1 - NMN adenyltransferase
MKKTFEQFRKSNLLFQVLKGPQHLECQKLFVLDSSFNPPHLAHFQLLSQTIKNFKLKDTRSHVLLLLAVNNADKLPKPASFPTRLEMMCLFADYLQEKLPQSVVSVGLTVFSKFIDKDKILHEQFVKGCSADIGYLVGFDTIARIFDEKYYHPLKISDVMESFMSGSQLYCLARGDCHLSAESQLRYASDILEGKFEPVIPREWGARIHVMQNDYPALRNVSSSEIRNKLKNGQVESLKDELPLCIYDYLINNKTIFD

SEQ ID NO: 54 - protein, 252 aa, Unknown
  NAMN adenyltransferase
  misc_feature (1)..(252): CAG84188 - NAMN adenyltransferase
MSLYTLVRAAATHSRPPAGTNFKAEIDDFMRGDERVRLLGDMPRKGSRVVVLDSSFNPPHYAHLELAMLGMTHTKDNCILLLLSITNADKKPAPAAFQQRLEMMELFKRSIDAEVVLGLTKEPYFVDKYKVIKRLLASHGLTPHLHFPMGLDTLVRLVDQKYYKEPVSEALKGFFQDCHVHVLTRDDIHFNMKELPEQWQRHIHMSKHSSKTDGVSSSNVRALVKSHKRHHEFERLVPPQILAYIVEHGLYK

SEQ ID NO: 55 - protein, 470 aa, Unknown
  NMN adenyltransferase
  misc_feature (1)..(470): CAG79991 - NMN adenyltransferase
MDPKLAPNFVRPSDKRPTAYRSPAPAQASLIPNIAPIQPYVLEDSQHEIDLPQDSPRLLPSRTNSRDSLVGLEQIALTLKHHSKHNPKDSNYHPAPVNIPRIHSELNMSDSSPDRCEKIDEVDLEVSPNEIQTNFSKFSLGDQALPPTTVEEAMAPTSPKSPKEPQVHTETGTADSSSRTPPYLLDNSGLRPIIQSADLEEVPIGVSRQARDLDHYKFPTHRLSEVMIEETKSPLVIVACGSFSPITYLHLRMFEMAMDSIREQTRFEVIGGYYSPVSDNYNKPGLAPAHHRVRMCELACERTSSWLMVDAWESLQPTYQRTATVLDHFNEEINIKRGGIKTVSGKRKGVKIMLLAGGDLIESMGEPNVWEERDLHHILGRYGCLIVERTGADVRSFLLSHDIMYEHRRNVLVIKQLIYNDISSTKVRLFIRRGMSVQYLIPNSVIRYIQEHRLYVGETEPVKQVLYDRE

SEQ ID NO: 56 - protein, 285 aa, Unknown
  NMN adenyltransferase
  misc_feature (1)..(285): An04g01700 - NMN adenyltransferase (POF1 homolog)
MTSPAFHALREKYAAGLRLFIASTRSLEVLASVPSGPPDPTSSTDVLYVLDSSFNPPTLAHSRIATSALLENPHKPSRLLLLLATQNADKPSKPAAFETRLAMMELFARDLLSSLQTNGTIPKAHNLPVIDIGVTKKPYFVDKAAEIERAGIYPQSLEQVHLTGYDTLIRIFNPKYYPPEHTLQPLGPFLSQHRLRVTMRPDSDWGSREEQEAFVADLAQGAMERVGGKREWADRIKLVEGKKPGAPSVSSTRARESAVANPQDLEWLVPPSVRDVILAEKPYTE

SEQ ID NO: 57 - protein, 281 aa, Unknown
  NMN adenyltransferase
  misc_feature (1)..(281): An11g06610 - NMN adenyltransferase (NMA2 homolog)
MTDTISDDVHRRSSPLPPAPMDDYAFPELRLKRTMDDPEKTPLLLVACGSFSPITYLHLRMFEMAADYVKFSTDFELVGGYLSPVSDAYRKAGLASAEHRVAMCQLAVDQTSDWLMVDTWEPMQKEYQPTAVVLDHFDHEINTVRQGIEAGNGTRKPIQIALLAGADLVHTMSTPGVWSEKDLDHILGKYGTFIVERTGTDIDEALAALQTWKKNIHVIQQLIQNDVSSTKIRLFLRRDMSVRYLIPVPVIHYIEQHNLYKDDSTASTDKGKGRQEPGSSG

SEQ ID NO: 58 - protein, 429 aa, Unknown
  Nicotinate phosphribosyltransferase
  misc_feature (1)..(429): Sc NPT1 - Nicotinate phosphribosyltransferase
MSEPVIKSLLDTDMYKITMHAAVFTNFPDVTVTYKYTNRSSQLTFNKEAINWLKEQFSYLGNLRFTEEEIEYLKQEIPYLPSAYIKYISSSNYKLHPEEQISFTSEEIEGKPTHYKLKILVSGSWKDTILYEIPLLSLISEAYFKFVDIDWDYENQLEQAEKKAETLFDNGIRFSEFGTRRRRSLKAQDLIMQGIMKAVNGNPDRNKSLLLGTSNILFAKKYGVKPIGTVAHEWVMGVASISEDYLHANKNAMDCWINTFGAKNAGLALTDTFGTDDFLKSFRPPYSDAYVGVRQDSGDPVEYTKKISHHYHDVLKLPKFSKIICYSDSLNVEKAITYSHAAKENGMLATFGIGTNFTNDFRKKSEPQVKSEPLNIVIKLLEVNGNHAIKISDNLGKNMGDPATVKRVKEELGYTERSWSGDNEAHRWT

SEQ ID NO: 59 - protein, 298 aa, Unknown
  CAG78849 - Nicotinate phosphoribosyltransferase
  misc_feature (1)..(298): CAG78849 - Nicotinate phosphoribosyltransferase
MTDTFDLSLKSANKISDKLPKELQNPQIGIVCGSGLGGLANALKAEPQVTIEYKDIPGFKVSTVAGHAGKLVVGLLGEKNVPVVCMVGRFHFYEGYDIQDTVFPIRVFSQIGIKTVILTNAAGGLNQDFKVGDIMLINDHINLPGLAGNNPLRGPNDEKFGVRFLPLSDAYDHDLRRAVYDIAKKQGVTRGIHEGTYAFVSGPTYESRAEARMLSTIGADAVGMSTVPEVIVARHCGIKVLALSLITNVVVLKKPDSALNDNAAKLDEGIADHSEVMEEGQRAADDLVGIVTDLVNVV

SEQ ID NO: 60 - protein, 414 aa, Unknown
  CAG82552 - Nicotinate phosphoribosyltransferase
  misc_feature (1)..(414): CAG82552 - Nicotinate phosphoribosyltransferase
MSTITSLLDTDLYKLTMQAAVLQHFPAAQATFLFKNRTPSKQLNDDAIEWLKSEIAALGELRFTEDEIVFLQKHVGFLPAEYFEYLKTCQLDPAAQVKVTVNTEGHLEIEVNGPWKDTILYEIPLLALVSEAYFKFVDKDWSYDGQSELAATKAQELIAQGCAFSEFGTRRRRSLKTHDIVIAGILEGLKSAQGNGIFTGTSNVYLAKKYNLKPIGTVAHEWMMGVAAATGDYSTANLRAMELWIQTVGDANAGVALTDTFGTESFLLDFNKPLTDIYNGVRQDSGDPLEYTKLLGDHYKQLGYEPMSKVIVYSDSLDVEKCGKYKAAAAENGLKAAFGVGTFFTNDFKRLSDGQKSTPLNIVIKIQQLNGQSCIKLSDNLSKNMGDPETVERVKRELGYVEKGDVIDESKRWN

SEQ ID NO: 61 - protein, 541 aa, Unknown
  An12g00760 - Nicotinate phosphoribosyl transferase (NPT1 homolog)
  misc_feature (1)..(541): An12g00760 - Nicotinate phosphoribosyl transferase (NPT1 homolog)
MGDFSSEYFPPNSSTCFILISHLWSLSSGPSFGIPLCALSFVLRCGPNYLVMAQNNTPPLPEGIFSLLDTDLYKLTMQCAVLKYFPDVYVTYGFTNRTPDMKLTRGAYKWLLAQLDALANIRVTNEEIEFLKKWCPYFNHAYLDYLTNLKLKPSEQIEVHFTPEQDTGSEDDLGNVNYVIKGLWVETILYEIPLLALTSQAYFMFTDKDWDYSCQEDKAFRKGCALLENGCIFSEFGSRRRRDYHTHDLVMQGLMQAAEEGKKQGWKGVFTGTSNVHFAMKYGVAPVGTVAHEWYMTIAAITDDYENANEMALRYWLGCFGEGVLGVALTDTFGTPAFLDAFRKPIPAFTSAGVGAVSTTASGPGTTTESAVQSEATTKPPIAAPLVNDAQGEHANKTYAQVYTGVRQDSGDPTYFVKMVRDFYDREGVKDPKTIVFSDSLDIEHCLEYKVIAEEAGFKPVFGVGTFFTNDFTNKSDGAKSKPLNIVIKIATANGRPAVKLSDNMGKNTGDKETVQSVKKKLGYVEHQWEEGDESNRWARK

SEQ ID NO: 62 - protein, 598 aa, Unknown
  Sc NRT1 - NR transport protein
  misc_feature (1)..(598): Sc NRT1 - NR transport protein
MSFSSIVSKFLRYLEIPAKNRTAVNFLRNPDLQPIKSANQTWGFWSNLAYWGAVSFTAGTWMSGSAALSVGLSYPETIVSFLLGNVLTIIFTMANSYPGYDWKIGFTLAQRFVFGIYGSAFGIIIRILMSIVNYGSNAWLGGLSINMILDSWSHHYLHLPNTLSPSVAMTTKQLVGFIIFHVLTALCYFMKPYHMNYLLIWSCVATCFAMLGIVIYLTKNAHGVGELFTSTKSTVTGSKRAWAWVYMISYWFGSISPGSTNQSDYSRFGSSNLAIWTGSVCALLIPATLVPIFGVISASTCDKLYGKQFWMPMDIFDYWLTNNYSAGARAGAFFCGLCFTMSQMSSTISNCGFATGMDMAGLLPKYVDIKRGALFCACISWACLPWNFYNSSSTFLTVMSSFGVVMTPIIAVMICDNFLIRKRQYSITNAFILKGEYYFTKGVNWRAIVAWVCGMAPGLPGIAWEVNNNYFHDSGIVKFFYGDSFFSFLISFFVYWGLCVFFPFKITVRHDDKDYYGAFTDEEARKKGMIPYSEISEEEIRAYTLGECYTTGHEYKPESSDNESPELIKTSSENTNVFEIVHQKDDEKHSFSTTQQVV

SEQ ID NO: 63 - protein, 592 aa, Unknown
  CAG80639 - NR transport protein
  misc_feature (1)..(592): CAG80639 - NR transport protein
MAHDSELELSDEKVVPSINQEKHSFFQRHLDNHPRMAQYNSQLQRFLKWIEVPTKEGEINTFLNNEDLKPVEVARQTWGWKNFVSFWIADSFNINTWEIAATGIQLGLTWWQVWLCVWIGYFFCGVFVVLSGRIGAIYHVSFPVAGRSTFGIFGSIWPVINRVVMACVWYGVQGWLGGQCIQVCLLAIWPSARHMKNGIPGSGTTTFEFLSYFLFWLFSLPFIYIRPHNLRHLFMVKAAIVPVAGISFLVWTCVKAHGIGPIMKQPATVHGSVMGWAFMTAIMNSLSNFATIIVNAPDFTRFAKEPNAIVLSQLIAVPTAFSLTSFIGIIVSSSATVLYDENIWNPLDVLHKFLEGNKSGSRAGVFFLGFAFAVAQLGTNIAANSLSAGTDMTALLPKYINIRRGGFICAGIALCICPWHLLSSSSNFTTYLSAYATFLSAIAGCSFSDYYLVRKGYIYVGDLYNASKGSTYMYRYGVNWRAFAAYFCGIAINVVGFADAVSDGGVNETARKMYQLNFFLGFLVSAISYYGFNWLSPVVGARETWSEDPNASAMYDEITTDELSQDSQSYDPEEWDRKIANDDPVKTTAIIS

SEQ ID NO: 64 - protein, 445 aa, Unknown
  An04g07070 - NR transport protein
  misc_feature (1)..(445): An04g07070 - NR transport protein
MDRIRGLIARRPTYEAIEQPVDADDESIRLTREEEQHPRFSKYEYAVFFLLGVSMLWAWNMFLAAAPYFYHRFHTDDWAATHYQPSILTVSTVTNLGSSFILAKLQKGASYPKRVTISLLINIVVFTLLAFSTILMKDVAVSTYFSFLMIMVFGASLATGINQNGVYAYVSGFGREEYTQAIMGGQGVAGVLPCIVQILSVVSVPSKKEGQKAPQESSKSAFAYFITATAVSSFALLAFLSLVKRRASSTLLDPTDDHSDPDVPENKSVSLWTLFKKLRFMASAIFLCFTVTMTFPVFTAEIESVHDTPGRSRLFDQAVFIPLAFFFWNAGDLMGRMLVLFPRLSLVNRPFVLFLFSIARAAFIPLYLLCNIRGRGAVVESDFFYLFIVQLLFGISNGYLGSNCMMGVGQWVSPDEREPAGGFMGLMLVGGLTAGSLMSFLVAGA

SEQ ID NO: 65 - protein, 340 aa, Unknown
  Sc URH1 - NR hydrolase
  misc_feature (1)..(340): Sc URH1 - NR hydrolase
MTVSKIPIWLDCDPGHDDAIAILLGCFHPAFNLLGISTCFGNAPPENTDYNARSLLTAMGKAQAIPVYKGAQRPWKREPHYAPDIHGISGLDGTSLLPKPTFEARTDKTYIEAIEEAILANNGEISFVSTGALTTLATVFRCKPYLKKSVKYISIMGGGLHGLGNCNPNLSAEFNVWIDPDAANYIFRDPDVKDKCIVVPLNLTHKAIATYKVNEMIYNEKNNSKLRELFLELFQFFAHTYKDMQGFESGPPIHDPVALMPLLEFYGWDPSSAVGFRYKRMDISCIDDVFNENSGKIIIEKEYPNDSDVGTIIGLDLNIQYFWDQIFEALNRADKMSTIG

SEQ ID NO: 66 - protein, 338 aa, Unknown
  CAG77762 - NR hydrolase
  misc_feature (1)..(338): CAG77762 - NR hydrolase
MIPIWLDCDPGHDDAVAILLAAKLPAFNLLGISTVFGNAPLEKTTVNAMAVLKAIGQDSEIKVYPGAERIQSRGGGGDGGSHDQSNQSHSPSSCSRGHYETAPSTFAPSIHGESGLDGTSLLPEITCTIEPHVDSTLVAMANAIRNNPGTCLVATGSLTNVSALFDRFPDVKNLVETVSIMGGGFGMGNWTPYAEFNIWCDAESASQVLGDPIVAAKVVLCPLNVTHTAIATEEVLSQIKSVGGNVSQMFFELMTFFKETYESEFGFKDGPPVHDPLSVAVLLNGIVTKERRVTVDVTVGGEQRGMLTEQVGGHLTVVEEVDLNKFWALVVTCLEACK

SEQ ID NO: 67 - protein, 435 aa, Unknown
  An18g04340 - NR hydrolase (URH1 homolog)
  misc_feature (1)..(435): An18g04340 - NR hydrolase (URH1 homolog)
MTLCPFRIRKVGLRADIAPCPLIRLIGTGFSYLQQSPQSPFRWHLGLPSFFSFIPSRSGFTMDSSVDTPIPLWLDCDPGHDDAFAILLAAHHPSLKLLGITTIHGNASLENTTINATRVLEAIGRPEIPVYPGSKKPFCRPALHAPNIHGDSGLDGTDLLPKASTAPITDKNPILAMRDALMAHPKGTPWVVATGTLTNVALLFATFPEVAEHIAGLTIMGGGVGEGFTDAPMSRLKGQENRIGNVTPLAEFNIYCDPEASQAIFSDPVVASKTTLITLDLTHQVLADQSVRNLVRHGVDDASVQPTTLRQMLYELLIFFANTYETVFGLSTGPPLHDPLAVAVILSTLNPGYAKNHPDQVLKFDDRGGERFSVNVITDGLHGTDIALVGSLGRSVVEANPNAVCIPRGVDLDAFWNMINDCIKRADECNAARKA

SEQ ID NO: 68 - protein, 240 aa, Unknown
  Sc NRK1 - NR kinase
  misc_feature (1)..(240): Sc NRK1 - NR kinase
MTSKKVILVALSGCSSSGKTTIAKLTASLFTKATLIHEDDFYKHDNEVPVDAKYNIQNWDSPEALDFKLFGKELDVIKQTGKIATKLIHNNNVDDPFTKFHIDRQVWDELKAKYDSINDDKYEVVIVDGFMIFNNTGISKKFDLKILVRAPYEVLKKRRASRKGYQTLDSFWVDPPYYFDEFVYESYRANHAQLFVNGDVEGLLDPRKSKNIKEFINDDDTPIAKPLSWVCQEILKLCKD

SEQ ID NO: 69 - protein, 254 aa, Unknown
  CAG83148 - NR kinase
  misc_feature (1)..(254): CAG83148 - NR kinase
MIRRKIAEKKLAKEEDKREVTLVALSGPSSSGKSTLARLLRDILPHVIIIHQDDFYLEDSQIPVIDGVQDWDCPEAFDFKLLSKVLSHVKQTGELPKNFKSKEDQNSLGPAALDENAVDAFKRRMHPYMPEFENKLIVILDGIMVYHDAQFTELFDIKILVRSSYENLKSRREARSGYVTLEGFWKDPEGYFHNIVWPGYLKTHKQLFENENPNGEPSKDATREGIRIVPTTDFDVAETLDWVFDVILDYYDLE

SEQ ID NO: 70 - protein, 281 aa, Unknown
  NR kinase
  misc_feature (1)..(281): An04g05800 - NR kinase (NRK1 homolog)
MTKSPLIPHPRDPTTQIQNWDTLSALDIPFLSASLSYIHAHGHLPPRLRSKEDLNDVAESGVSEEVVSRMREVVRGRVPSYFLEDNTNSVSGDGVRTIVFLEGFLLFAPPKEEDPRHGLRDVQEKMDVRLFLPARYDNVKERREGRSGYVTIGPAPVQQGGGGGTELPQRGSVEVDLEEEDDRPPQSFWEDPPGYVDDIVWPNYVQDHAWLLLPEGEEGSAQTAWKDADTQELVRLVGQGVNLRLDTGVTVAPGQGKASMTDILEWAVEEVLKHIAETEGQ

SEQ ID NO: 71 - protein, 311 aa, Unknown
  NMN/purine phosphorylase
  misc_feature (1)..(311): Sc PNP1 - NMN/purine phosphorylase
MSDILNVSQQREAITKAAAYISAILEPHFKNTTNFEPPRTLIICGSGLGGISTKLSRDNPPPVTVPYQDIPGFKKSTVPGHSGTLMFGSMNGSPVVLMNGRLHGYEGNTLFETTFPIRVLNHMGHVRNLIVTNAAGGINAKYQACDLMCIYDHLNIPGLAGQHPLRGPNLDEDGPRFLALSDAYDLELRKLLFKKWKELKIQRPLHEGTYTFVSGPTFETRAESKMIRMLGGDAVGMSTVPEVIVARHCGWRVLALSLITNTCVVDSPASALDESPVPLEKGKATHAEVLENGKIASNDVQNLIAAVMGEL

SEQ ID NO: 72 - protein, 298 aa, Unknown; identical to SEQ ID NO: 59
  NMN/purine phosphorylase
  misc_feature (1)..(298): CAG78849 - NMN/purine phosphorylase

SEQ ID NO: 73 - protein, 550 aa, Escherichia coli
  misc_feature (1)..(550): Escherichia coli UshA (nucleotide hydrolase)>gi|16128464|ref|NP_415013.1| bifunctional UDP-sugar hydrolase/5′-nucleotidase [Escherichia coli str. K-12 substr. MG1655]
MKLLQRGVALALLTTFTLASETALAYEQDKTYKITVLHTNDHHGHFWRNEYGEYGLAAQKTLVDGIRKEVAAEGGSVLLLSGGDINTGVPESDLQDAEPDFRGMNLVGYDAMAIGNHEFDNPLTVLRQQEKWAKFPLLSANIYQKSTGERLFKPWALFKRQDLKIAVIGLTTDDTAKIGNPEYFTDIEFRKPADEAKLVIQELQQTEKPDIIIAATHMGHYDNGEHGSNAPGDVEMARALPAGSLAMIVGGHSQDPVCMAAENKKQVDYVPGTPCKPDQQNGIWIVQAHEWGKYVGRADFEFRNGEMKMVNYQLIPVNLKKKVTWEDGKSERVLYTPEIAENQQMISLLSPFQNKGKAQLEVKIGETNGRLEGDRDKVRFVQTNMGRLILAAQMDRTGADFAVMSGGGIRDSIEAGDISYKNVLKVQPFGNVVVYADMTGKEVIDYLTAVAQMKPDSGAYPQFANVSFVAKDGKLNDLKIKGEPVDPAKTYRMATLNFNATGGDGYPRLDNKPGYVNTGFIDAEVLKAYIQKSSPLDVSVYEPKGEVSWQ

SEQ ID NO: 74 - protein, 1462 aa, Bacillus subtilis
  misc_feature (1)..(1462): Bacillus subtilis YfkN (nucleotide hydrolase) >gi|16077851|ref|NP_388665.1| nucleotide phosphoesterase [Bacillus subtilis subsp. subtilis str. 168]
MRIQKRRTHVENILRILLPPIMILSLILPTPPIHAEESAAPQVHLSILATTDIHANMMDYDYYSDKETADFGLARTAQLIQKHREQNPNTLLVDNGDLIQGNPLGEYAVKYQKDDIISGTKTHPIISVMNALKYDAGTLGNHEFNYGLDFLDGTIKGADFPIVNANVKTTSGENRYTPYVINEKTLIDENGNEQKVKVGYIGFVPPQIMTWDKKNLEGQVQVQDIVESANETIPKMKAEGADVIIALAHTGIEKQAQSSGAENAVFDLATKTKGIDAIISGHQHGLFPSAEYAGVAQFNVEKGTINGIPVVMPSSWGKYLGVIDLKLEKADGSWKVADSKGSIESIAGNVTSRNETVTNTIQQTHQNTLEYVRKPVGKTEADINSFFAQVKDDPSIQIVTDAQKWYAEKEMKDTEYKNLPILSAGAPFKAGGRNGANYYTNIPAGDLAIKNVGDLYLYDNTVQIVKLTGSEVKDWLEMSAGQFNQIDPAKGGDQALLNENFRSYNFDVIDGVTYQVDVTKPAKYNENGKVINADSSRIINLSYEGKPISPSQEFLVVTNNYRASGGGGFPHLTSDKIVHGSAVENRQVLMDYIIEQKTVNPKADNNWSIAPVSGTNLTFESSLLAKPFADKADDVAYVGKSANEGYGVYKLQFDDDSNPDPPKDGLWDLTVMHTNDTHAHLDDAARRMTKINEVRSETNHNILLDAGDVFSGDLYFTKWNGLADLKMMNMMGYDAMTFGNHEFDKGPTVLSDFLSGNSATVDPANRYHFEAPEFPIVSANVDVSNEPKLKSFVKKPQTFTAGEKKEAGIHPYILLDVDGEKVAVFGLTTEDTATTSSPGKSIVFNDAFETAQNTVKAIQEEEKVNKIIALTHIGHNRDLELAKKVKGIDLIIGGHTHTLVDKMEVVNNEEPTIVAQAKEYGQFLGRVDVAFDEKGVVQTDKSNLSVLPIDEHTEENPEAKQELDQFKNELEDVKNEKVGYTDVALDGQREHVRTKETNLGNFIADGMLAKAKEAAGARIAITNGGGIRAGIDKGDITLGEVLNVMPFGNTLYVADLTGKQIKEALEQGLSNVENGGGAFPQVAGIEYTFTLNNKPGHRVLEVKIESPNGDKVAINTDDTYRVATNNFVGAGGDGYSVFTEASHGEDLGYVDYEIFTEQLKKLGNKVSPKVEGRIKEVFLPTKQKDGSWTLDEDKFAIYAKNANTPFVYYGIHEGSQEKPINLKVKKDQVKLLKERESDPSLTMFNYWYSMKMPMANLKTADTAIGIKSTGELDVSLSDVYDFTVKQKGKEIKSFKEPVQLSLRMFDIEEAHNPAIYHVDRKKKAFTKTGHGSVDDDMVTGYTNHFSEYTILNSGSNNKPPAFPSDQPTGGDDGNHGGGSDKPGGKQPTDGNGGNDTPPGTQPTNGSGGNGSGGSGTDGPAGGLLPDTATSMYSILLAGFLISALGTAMYLHQRRKQNRANQA

SEQ ID NO: 75 - protein, 694 aa, Corynebacterium glutamicum
  misc_feature (1)..(694): Corynebacterium glutamicum Cg0397 (nucleotide hydrolase) >gi|41324559|emb|CAF18899.1| PROBABLE 5′-NUCLEOTIDASE PRECURSOR [Corynebacterium glutamicum ATCC 13032]
MKRLSRAALAVVATTAVSFSALAVPAFADEASNVELNILGVTDFHGHIEQKAVKDDKGVITGYSEMGASGVACYVDAERADNPNTRFITVGDNIGGSPFVSSILKDEPTLQALSAIGVDASALGNHEFDQGYSDLVNRVSLDGSGSAKFPYLGANVEGGTPAPAKSEIIEMDGVKIAYVGAVTEETATLVSPAGIEGITFTGDIDAINAEADRVIEAGEADVVIALIHAEAAPTDLFSNNVDVVFSGHTHFDYVAEGEARGDKQPLVVIQGHEYGKVISDVEISYDREAGKITNIEAKNVSATDVVENCETPNTAVDAIVAAAVEAAEEAGNEVVATIDNGFYRGADEEGTTGSNRGVESSLSNLIAEAGLWAVNDATILNADIGIMNAGGVRADLEAGEVTFADAYATQNFSNTYGVREVSGAQFKEALEQQWKETGDRPRLALGLSSNVQYSYDETREYGDRITHITFNGEPMDMKETYRVTGSSFLLAGGDSFTAFAEGGPIAETGMVDIDLFNNYIAAHPDAPIRANQSSVGIALSGPAVAEDGTLVPGEELTVDLSSLSYTGPEAKPTTVEVTVGTEKKTADVDNTIVPQFDSTGKATVTLTVPEGATSVKIATDNGTTFELPVTVNGEGNNDDDDDKEQQSSGSSDAGSLVAVLGVLGALGGLVAFFLNSAQGAPFLAQLQAMFAQFM

SEQ ID NO: 76 - protein, 347 aa, Escherichia coli
  misc_feature (1)..(347): Escherichia coli NadA >gi|260450103| gb|ACX40525.1| quinolinate synthetase complex, A subunit [Escherichia coli DH1]
MSVMFDPDTAIYPFPPKPTPLSIDEKAYYREKIKRLLKERNAVMVAHYYTDPEIQQLAEETGGCISDSLEMARFGAKHPASTLLVAGVRFMGETAKILSPEKTILMPTLQAECSLDLGCPVEEFNAFCDAHPDRTVVVYANTSAAVKARADWVVTSSIAVELIDHLDSLGEKIIWAPDKHLGRYVQKQTGGDILCWQGACIVHDEFKTQALTRLQEEYPDAAILVHPESPQAIVDMADAVGSTSQLIAAAKTLPHQRLIVATDRGIFYKMQQAVPDKELLEAPTAGEGATCRSCAHCPWMAMNGLQAIAEALEQEGSNHEVHVDERLRERALVPLNRMLDFAATLRG

SEQ ID NO: 77 - protein, 368 aa, Bacillus subtilis
  misc_feature (1)..(368): Bacillus subtilis NadA >gi|16079837|ref |NP_390663.1| quinolinate synthase A [Bacillus subtilis subsp. subtilis str. 168]
MSILDVIKQSNDMMPESYKELSRKDMETRVAAIKKKFGSRLFIPGHHYQKDEVIQFADQTGDSLQLAQVAEKNKEADYIVFCGVHFMAETADMLTSEQQTVVLPDMRAGCSMADMADMQQTNRAWKKLQHIFGDTIIPLTYVNSTAEIKAFVGKHGGATVTSSNAKKVLEWAFTQKKRILFLPDQHLGRNTAYDLGIALEDMAVWDPMKDELVAESGHTNVKVILWKGHCSVHEKFTTKNIHDMRERDPDIQIIVHPECSHEVVTLSDDNGSTKYIIDTINQAPAGSKWAIGTEMNLVQRIIHEHPDKQIESLNPDMCPCLTMNRIDLPHLLWSLEQIEKGEPSGVIKVPKAIQEDALLALNRMLSIT

SEQ ID NO: 78 - protein, 428 aa, Corynebacterium glutamicum
  misc_feature (1)..(428): Corynebacterium glutamicum NadA >gi|41325294|emb|CAF19774.1| QUINOLINATE SYNTHETASE [Corynebacterium glutamicum ATCC 13032]
MTTSITPSVNLALKNANSCNSELKDGPWFLDQPGMPDVYGPGASQNDPIPAHAPRQQVLPEEYQRASDDELHRRIREAKDTLGDKVVILGHFYQRDEVIQHADFVGDSFQLARAAKTRPEAEAIVFCGVHFMAETADLLSTDEQSVILPNLAAGCSMADMADLDSVEDCWEQLTSIYGDDTLIPVTYMNSSAALKGFVGEHGGIVCTSSNARSVLEWAFERGQRVLFFPDQHLGRNTAKAMGIGIDQMPLWNPNKPLGGNTVSELENAKVLLWHGFCSVHKRFTVEQINKARAEYPDVHVIVHPESPMPVVDAADSSGSTDFIVKAIQAAPAGSTFAIGTEINLVQRLAAQYPQHTIFCLDPVICPCSTMYRIHPGYLAWALEELVAGNVINQISVSESVAAPARVALERMLSVVPAAPVTPSSSKDA

SEQ ID NO: 79 - DNA, 20 bp, Artificial Sequence
  primer 10444
cggtaagtcccgtctagcct

SEQ ID NO: 80 - DNA, 25 bp, Artificial Sequence
  primer 10447
atgtttgcaaaacgattcaaaacct

SEQ ID NO: 81 - DNA, 37 bp, Artificial Sequence
  primer 11222
ttacaccgaatttctaataataaccgggcaggccatg

SEQ ID NO: 82 - DNA, 37 bp, Artificial Sequence
  primer 11223
ggcctgcccggttattattagaaattcggtgtaagag

SEQ ID NO: 83 - DNA, 40 bp, Artificial Sequence
  primer 11226
cttttacaccgaatttttaataataaccgggcaggccatg

SEQ ID NO: 84 - DNA, 37 bp, Artificial Sequence
  primer 11227
ggcctgcccggttattattaaaaattcggtgtaaaag

SEQ ID NO: 85 - DNA, 40 bp, Artificial Sequence
  primer 11230
cggatgaagcggaatgttaataataaccgggcaggccatg

SEQ ID NO: 86 - DNA, 37 bp, Artificial Sequence
  primer 11231
ggcctgcccggttattattaacattccgcttcatccg

SEQ ID NO: 87 - DNA, 38 bp, Artificial Sequence
  primer 11232
gaaaggtggtgaactactatgaaaacagcagcatacgc

SEQ ID NO: 88 - DNA, 40 bp, Artificial Sequence
  primer 11233
gcgtatgctgctgttttcatagtagttcaccacctttctc

SEQ ID NO: 89 - DNA, 40 bp, Artificial Sequence
  primer 11234
cacttacaccgaacttctaataataaccgggcaggccatg

SEQ ID NO: 90 - DNA, 37 bp, Artificial Sequence
  primer 11235
ggcctgcccggttattattagaagttcggtgtaagtg

SEQ ID NO: 91 - DNA, 33 bp, Artificial Sequence
  primer 11342
aagggaggtttcatatgaaaattgttaaagatt

SEQ ID NO: 92 - DNA, 37 bp, Artificial Sequence
  primer 11351
tttaacaattttcatatgaaacctcccttaattctcg

SEQ ID NO: 93 - DNA, 35 bp, Artificial Sequence
  primer 11351
gtgaactactatgaaaattgtaaaaaactttattg

SEQ ID NO: 94 - DNA, 35 bp, Artificial Sequence
  primer 11352
ttacaattttcatagtagttcaccacctttctcta

SEQ ID NO: 95 - DNA, 35 bp, Artificial Sequence
  primer 11353
aagggaggtttcatatgaaaatcgttaaagacttc

SEQ ID NO: 96 - DNA, 35 bp, Artificial Sequence
  primer 11354
taacgattttcatatgaaacctcccttaattctcg

SEQ ID NO: 97 - DNA, 36 bp, Artificial Sequence
  primer 11159
gctacttactctcgagttactttttccagaaatcat

SEQ ID NO: 98 - DNA, 33 bp, Artificial Sequence
  primer 11160
gctaacttagcatatgatgacattgcaacaaca